(19) United States
(12) Patent Application Publication
(10) Pub. No.: US 2016/0243277 A1
(43) Pub. Date: Aug. 25, 2016

(54) FAST CURING POROUS MATERIALS AND CONTROL THEREOF

(71) Applicant: THE TEXAS A & M UNIVERSITY SYSTEM, College Station, TX (US)

(72) Inventors: Elizabeth M. Cosgriff-Hernandez, College Station, TX (US); Robert Scott Moglia, College Station, TX (US)

(73) Assignee: The Texas A & M University System, College Station, TX (US)

(21) Appl. No.: 15/024,335

(22) PCT Filed: Sep. 23, 2014
(86) PCT No.: PCT/US14/57025

§ 371 Date: Mar. 23, 2016

Related U.S. Application Data
(60) Provisional application No. 61/881,406, filed on Sep. 23, 2013.

(51) Int. Cl. A61L 27/16; A61L 27/54; A61L 27/56

(52) U.S. Cl. CPC A61L 27/16; A61L 27/56; A61L 27/54; A61L 2300/254; A61L 2300/414; A61L 2300/252

(57) ABSTRACT

High internal phase emulsions (HIPEs) and polyHIPEs formed therefrom. HIPE components have been adjusted to delay and/or prevent polymerization and free radical decomposition at certain temperatures. Methods include preparing two water-in-oil emulsions; one emulsion includes an oxidizing initiator at a concentration not sufficient to induce polymerization, and another emulsion includes a reducing agent. The two emulsions may be stored for months. When combined, a redox reaction is induced, resulting in polymerization (polyHIPE formation) at temperatures below 100° C., such as between 20° C. and 40° C. This allows a final composition to be formed in situ, with or without highly sensitive materials, including biologically-derived or active constituents (e.g., cells, growth factors, enzymes, proteins, drugs, chemoactive agents). The polymerization rate may be short, as fast as thirty seconds. HIPE components as well as the redox reaction may be controlled to manipulate time to cure, pore size, distribution of pores, and/or internal contents.

### CROSS-REFERENCE TO RELATED APPLICATION

This application claims the benefit of U.S. Provisional Patent Application No. 61/881,406 filed Sep. 23, 2013, the entirety of which is incorporated herein by reference.

### STATEMENT REGARDING FEDERALLY SPONSORED RESEARCH/DEV

The disclosed was made with government support under 1R21AR057531 awarded by the National Institutes of Health. The government has certain rights in the invention.

### TECHNICAL FIELD

As disclosed herein are fast curing high porosity materials and methods of making said materials, including controlling their time to cure and their porosity in order to optimize their ex vivo, in vivo and/or in situ delivery.

### BACKGROUND

The controlled delivery of a fast curing resin, particularly a resin suitable for in vivo use, remains challenging. First, the rate of polymerization and cross-linking as well as the method of delivery hampers the use of many polymeric resin materials. In addition, while some polymers offer structural benefits, they have been found unsuitable for use in vivo because of their chemical make-up or because of the complicated or time consuming manner of processing that is required prior to delivery. For example, many polymeric resins require complicated steps during or after processing and before they are suitable for delivery. Many polymeric resins have only a very short shelf-life. Many polymeric resins offer only limited structural support following polymerization and many are not specifically designed to be degradable after delivery. Many structural tissues or organs, however, require that any polymeric material incorporated therein offer adequate mechanical properties (to withstand physiologic loading, especially when tissue function is not yet restored), as well as an adequate internal morphology, preferably an interconnected network, that promotes cell growth, allows sufficient nutrient and/or waste transport and neovascularization. Unfortunately, many polymeric resins require either toxic diluents and/or high cure temperatures for cross-linking and curing, making them unsuitable for controlled in vivo delivery. Some polymeric resins, including certain gel or suspension systems, while capable of polymerizing in vivo remain difficult to control their time to cure or their porosity, or internal morphology, or mechanical strength.

Many current biodegradable polymer formulations require roughly two hours or more to cure at body temperature. In practice, however, a fast-curing material that reduces surgical time, lowers the patient's risk of infection, and rapidly stabilizes defects is preferred. Poly(methyl methacrylate) (PMMA) bone cement, which is not biodegradable, but capable of curing in about 15 minutes, is the most common injectable system used clinically to stabilize orthopedic implants. PMMA, however, does not facilitate tissue regeneration because it is highly exothermic, non-degradable, and non-porous. Previous iterations of high internal phase polymeric resin compositions relied on thermal initiation, the rate of which increases exponentially with temperature. For in situ curing, however, the physiologic temperature of the body constrains the cure temperature to 37° C. This temperature is well below the typical temperature of most thermal initiators. When some high internal phase polymeric resin compositions tried to use non-thermal initiators, the cure systems were instead found to contain toxic or non-degradable components.

There remains a need for a polymeric material that is hardenable in a controlled setting and capable of being delivered site specifically in vivo, such that it can be cured in a controlled manner and when cured is suitably biocompatible (in structure and morphology). Current polymeric systems have not been found to meet said needs, nor are they capable of being predesigned to offer desired properties upon delivery, properties that include morphologic, mechanical, and structural, as well as an ability to withstand physiologic compressive stresses.

### SUMMARY

Described herein are high internal phase resin compositions comprising biocompatible polymeric materials. The high internal phase resins when combined are capable of meeting the needs described above. The high internal phase resins described herein can be prepared to polymerize and cure in a controlled manner. The high internal phase resins when combined are capable of polymerizing and curing in an in vivo environment. In some embodiments, the high internal phase resins when combined are capable of polymerizing and curing in situ. The high internal phase resins when combined may be delivered site specifically and under controlled design conditions. The high internal phase resins when combined may be pre-selected to control the rate of polymerization or time to cure. The high internal phase resins when combined may be pre-selected to control the degree and extent of porosity. The high internal phase resins, when combined, are capable of forming a three-dimensional highly porous architecture. The formed three-dimensional architecture is a monolith. The formed three-dimensional architecture is a rigid structure having a foam body. The formed three-dimensional architecture is biocompatible, sufficiently porous and also capable of withstanding physiologic compressive stresses when fully cured. The formed three-dimensional architecture may be biodegradable. The three-dimensional architecture may contain one or more materials or active agents when formed. The formed three-dimensional architecture may be specifically fabricated and/or shaped to suit a particular in vivo environment. The formed three-dimensional architecture may be specifically fabricated to introduce one or more material or active agents into its environment.

One or more of the high internal phase resins described herein are also stable as emulsions (without fully polymerizing) enabling a long shelf-life prior to delivery or use. Said high internal phase resins may be pre-selected and tuned to control the rate of polymerization or time to cure of a final three-dimensional monolith.

In one or more forms is a first high internal phase resin composition. The first high internal phase resin is formed as a water-in-oil emulsion comprising at least a first biodegradable polymeric material in a continuous (oil) phase and an oxidizing initiator in either the continuous phase or a dispersed (water) phase. The water-in-oil high internal phase emulsion has a water volume of at least about 75% or greater. The first high internal phase resin emulsion may be designed to be stable as an emulsion without fully polymerizing at ambient or room temperature. In one or more embodiments, the first high internal phase resin is stable as an emulsion (without fully polymerizing) for at least one or more hours or two or more hours or up to about 12 hours or up to or greater than 24 hours, such as at an ambient temperature, or up to at least six months or more, such as at a temperature less than an ambient temperature. The oxidizing initiator is selected and in an amount that does not promote polymerization or delays polymerization of the first high internal phase resin. In one or more embodiments, the oxidizing initiator is selected and in an amount that is low enough to prevent polymerization or delay polymerization for at least one or more hours or two or more hours or up to about 12 hours or up to or greater than 24 hours or up to at least six months or more, such as when the first high internal phase resin emulsion at an ambient temperature, is at a sub ambient temperature, or is at a reduced temperature, such as about 4 degrees C. or less than 4 degrees C. The oxidizing initiator may be a nonaqueous free-radical oxidizing agent. The oxidizing initiator may be an aqueous free-radical oxidizing agent. Exemplary oxidizing initiators include but are not limited to peroxides, persulfates, and azo initiators. The first high internal phase resin composition may also include in the dispersed phase at least one chemical that prevents Ostwald ripening. The first high internal phase resin composition is emulsified by mechanical dispersion of the components described. The first high internal phase resin emulsion is stable in a relatively unpolymerized emulsified state at ambient or room temperature or at a temperature below ambient or room temperature.

The first biodegradable polymeric material of the first high internal phase resin emulsion is stabilized with an emulsifier in the continuous phase. The emulsifier may be an amphiphilic surfactant having a polar, water-soluble head group attached to a nonpolar, water-insoluble hydrocarbon chain. The emulsifier is selected as one lacking hydrogen bond donor sites (donors) in its hydrophilic, polar head region. In one or more embodiments, the emulsifier has a hydrophilic-lipophilic balance (HLB) in a range of between about 2 and about 9. In some embodiments, the emulsifier has a hydrophilic-lipophilic balance (HLB) in a range of between about 3 and about 5.

In a second high internal phase resin composition are components that form a water-in-oil emulsion, the components including at least a second biodegradable polymeric material in the continuous (oil) phase and a reducing agent in the dispersed (water) phase or in the continuous phase. Said water-in-oil high internal phase emulsion has a water volume of at least about 75% or greater. The second high internal phase resin emulsion may be designed to be stable as an emulsion without fully polymerizing at ambient or room temperature. In one or more embodiments, the second high internal phase resin is stable as an emulsion (without fully polymerizing) for at least one or more hours or two or more hours or up to about 12 hours or up to or greater than 24 hours, such as at an ambient temperature, or up to at least six months or more, such as at a temperature less than an ambient temperature. The reducing agent is in an amount that does not promote polymerization or delays polymerization of the second high internal phase resin. The reducing agent is selected from one or more of a hydrocarbon, metal ion, vitamin, bioactive agent and the like. The reducing agent is selected and in an amount that is low enough to prevent polymerization or delay polymerization for at least one or more hours or two or more hours or up to about 12 hours or up to or greater than 24 hours or up to at least six months or more, such as when the first high internal phase resin emulsion at an ambient temperature, is at a sub ambient temperature, or is at a reduced temperature, such as about 4 degrees C. or less than 4 degrees C. The second high internal phase resin may also include in the dispersed phase at least one chemical that prevents Ostwald ripening. The second high internal phase resin composition is emulsified by mechanical dispersion of the components described. The second high internal phase resin emulsion is stable in a relatively unpolymerized emulsified state at ambient or room temperature or at a temperature below ambient or room temperature.

The second biodegradable polymeric material of the second high internal phase emulsion is stabilized with an emulsifier in the continuous phase. The emulsifier may be an amphiphilic surfactant having a polar, water-soluble head group attached to a nonpolar, water-insoluble hydrocarbon chain. The emulsifier is selected as one lacking hydrogen bond donor sites (donors) in its hydrophilic, polar head region. The emulsifier has, in one or more embodiments, a hydrophilic-lipophilic balance (HLB) in a range of between about 2 and about 9. In additional embodiments, the emulsifier has a hydrophilic-lipophilic balance (HLB) in a range of between about 3 and about 5.

The emulsifier stabilizing the second biodegradable polymeric material may be essentially the same as the emulsifier stabilizing the first biodegradable polymeric material. The emulsifier stabilizing the second biodegradable polymeric material may also be different from the emulsifier stabilizing the first biodegradable polymeric material.

The first and the second biodegradable polymeric material are each selected for polymerization of a porous polymer (also referred to as polyHIPE), and will include: (a) a macromonomer (macromer) having at least one reactive end group, which is biodegradable, and having a suitable viscosity for emulsion in water; and (b) reaction thermodynamics that allow polymerization and/or curing at a physiologic condition. The at least one reactive end group will crosslink the macromer at a thermal or ambient or a physiologic temperature. The molecular weight of the biodegradable polymeric material will assist in maintaining a viscosity, and preferably a controlled viscosity that replaces (and no longer requiring) a toxic diluent for macromer stabilization. The at least one reactive end group has at least one unsaturated (double) bond for undergoing free radical cross linking. In one or more embodiments, the at least one reactive end group may be an acrylate end group or a methacrylate end group.

The macromer for the first biodegradable polymeric material and the second biodegradable polymeric material will have a selected viscosity and hydrophobicity. In one or more embodiments, the hydrophobicity, determined by an octanol-water partition coefficient (LogP), is greater than 2. In some embodiments, the LogP is in a range of between 2 and 8. In some embodiments, the LogP is in a range of between 2 and 4. The macromer preferably is but is not required to have a viscosity in a range that is near that of water, which is 1 cP. In some embodiments, the viscosity is in a range of between about 0.08 cP and about 1000 cP. In some embodiments, the viscosity is not greater than 0.150 cP.

Suitable macromers are ones prepared from an ester based monomer, macromer, and/or polymer or from an anhydride based monomer, macromer, and/or polymer. A suitable macromer is represented by but is not limited to a biodegradable polymer having one or more ester linkages or a biodegradable polymer having one or more anhydride linkages. Exemplary embodiments include a biodegradable fumarate based polymer having one or more ester linkages (e.g., propylene fumarate dimethacrylate [PFDMA]), a biodegradable diglycol based polymer having one or more ester linkages (e.g., ethylene glycol dimethacrylate [EGDMA]), a butane diol dimethacrylate [BDMA]) and a biodegradable acrylic based polymer having one or more anhydride linkages (e.g., methacrylic anhydride [MA]).

In some embodiments, the first biodegradable polymeric material and the second biodegradable polymeric material are the same. In some embodiments, the first biodegradable polymeric material and the second biodegradable polymeric material are similar, having the same or similar linkages and/or end groups.

Described herein is also a third high internal phase resin composition. The third high internal phase resin composition is formed as a third emulsion by combining the first high internal phase resin emulsion previously described and the second high internal phase resin emulsion previously described. The first high internal phase resin emulsion and the second high internal phase resin emulsion are combined in a mixing chamber or apparatus, thereby providing the third high internal phase resin composition. The third high internal phase resin emulsion when formed is capable of polymerizing, serving as a template for obtaining a porous polymer from high internal phase emulsions (polyHIPE). The third high internal phase resin emulsion will undergo a redox reaction and polymerization of the continuous phase. The third high internal phase resin emulsion will undergo a free radical cross-linking of the reactive end groups. The third high internal phase emulsion may be a foam emulsion prior to curing. The third high internal phase resin emulsion may be incubated or otherwise controlled to modify the degree of crosslinking, the rate of polymerization, and the extent of porosity or three-dimensional architecture.

In one or more embodiments, the third high internal phase resin composition is formed by combining the first high internal phase resin emulsion and the second high internal phase resin emulsion in a mixing chamber for polymerization and free radical cross-linking, and thereafter introducing after combining to a mold followed by providing the molded composition either when partially or fully set or cured to a location in vivo. In additional embodiments, the first and second high internal phase resins may be combined in a mixing chamber for polymerization and free radical cross-linking, and thereafter introduced to a location in vivo. The mixing may be by mechanical dispersion or by others means for blending the first and second high internal phase emulsions. The mixing may include incubating at a temperature below or well below 100 degrees Centigrade during or upon blending. The mixing may include incubating at a more physiologic temperature below, such as between about 25 degrees and 45 degrees Centigrade. In various embodiments, blending of the first and second high internal phase emulsions in a chamber provides a third emulsion that has a foam body. The foam body has a three-dimensional architecture that is highly porous. The foam body is prepared in the absence of a blowing agent. The foam body may incubate for a period of time. Pore size and pore distribution of the foam body may be controlled by one or more of the following, as examples: mixing condition, emulsifier in the first high internal phase resin emulsion, emulsifier in the second high internal phase resin emulsion, the type and amount of polymerization and free radical cross-linking agents, and polymerization temperature. In one or more embodiments, decreasing the overall emulsifier concentration increases the average pore diameter in the foam body. In various embodiments, decreasing the rate of mixing in the mixing chamber increases the average pore diameter in the foam body. In various embodiments, increasing the temperature for making the foam body increases the average pore diameter in the foam body. In various embodiments, increasing the temperature for curing (incubating) the foam body increases the average pore diameter in the foam body. The foam body will often have an overall porosity of about or greater than 75%. In some embodiments, the foam body when fully cured (to its hardened state) will have an average pore size (as a cross-sectional diameter) in a range from at least about 2 μm to about 50 μm. In some embodiments, the foam body when fully cured (to its hardened state) will have an average compressive modulus of at or about 2-50 MPa and strength of at or about 1-10 MPa.

The first high internal phase resin emulsion and the second high internal phase resin emulsion are combined to form the third high internal phase emulsion at a selected temperature. The third high internal phase emulsion is mixed at a selected temperature. In some embodiments, the third high internal phase emulsion is incubated at a selected temperature. The combining temperature and/or the mixing temperature and/or the incubating temperature may be the same. In one or more embodiments, said temperature is a low cure temperature. In various embodiments, the temperature is 100° C. or less. In various embodiments, the temperature is a physiologic temperature. In various embodiments, the temperature is between about 15° C. and about 55° C. In various embodiments, the temperature is between about 20° C. and about 45° C. Upon combining the first and second high internal phase resin emulsions, the rate or time to cure of the third high internal phase emulsion decreases with increasing temperature.

The first and second high internal phase resin emulsions when combined as a third high internal phase emulsion promote initiator decomposition and free radical production therein. The free radical production is provided at a thermal, ambient or physiologic temperature. In one or more embodiments, cure time of the third high internal phase resin emulsion is controlled by adjusting the amount of free radical production, whereby an increase in the production (concentration) of free radicals increases the rate of polymerization (and cross-linking) and reduces the cure time or set time that follows. Free radical production may be tuned by controlling the reducing agent or the ratio of reducing agent to oxidizing initiator, whereby the rate of decomposition of the oxidizing initiator is increased in the presence of the reducing agent. The presence of the reducing agent reduces the overall concentration of an initiator required in the third high internal phase emulsions as compared with alternative high internal phase emulsions that lack a reducing agent and initiate cross-linking only in the presence of an initiator alone.

In one or more embodiments, the first high internal phase resin emulsion and the second high internal phase resin emulsion have similar components, excluding the oxidizing initiator in the first high internal phase resin emulsion and the reducing agent in the second high internal phase resin emulsion, such that only the oxidizing initiator in the first high internal phase resin emulsion is replaced by a reducing agent in the second high internal phase resin emulsion.

Even further as described herein is a resulting emulsion body, provided as a three-dimensional monolith, suitable for use in vivo, ex vivo and/or in situ. The resulting emulsion body is prepared from two independently prepared high internal phase resin emulsions, one of which contains a free radical oxidizing agent and another one that contains a reducing agent. The resulting emulsion body is prepared by a redox reaction between a first high internal phase resin emulsion containing a free radical oxidizing agent in an amount that delays or prevents cross-linking and a second high internal phase resin emulsion containing a reducing agent in an amount that delays or prevents cross-linking. In one or more embodiments, the three-dimensional monolith is a foam body when cured. It may be operable for tissue replacement and stabilization. In one or more embodiments, the three-dimensional monolith when cured may be operable for stabilization as a soft tissue. In another embodiment, the three-dimensional monolith when cured may be operable for stabilization as a hard tissue. In still another embodiment, the three-dimensional monolith when cured may be operable for stabilization as an interface tissue. In yet a further embodiment, the three-dimensional monolith when cured may be operable as a catalyst support. In yet another embodiment, the three-dimensional monolith when cured may be operable as a component in a system sensitive to heat or solvents. In a further embodiment, the three-dimensional monolith may be biodegradable. In some embodiments, the three-dimensional monolith when formed will degrade at a rate complementary with tissue regeneration.

A high internal phase emulsion when formed may comprise a biodegradable polymeric material, the biodegradable polymeric material comprising: at least one end group selected from an acrylate and a methacrylate; and one or more linkages selected from an anhydride and an ester. The biodegradable polymeric material may have an octanol-water partition coefficient of between about 2 and about 8 and a viscosity of between about 0.08 cP and about 1000 cP. The biodegradable polymeric material is generally stabilized with a quantity of an emulsifier lacking hydrogen bond donors in its hydrophilic head region while having a hydrophilic-lipophilic balance in a range of between about 2 and about 9. The high internal phase emulsion further comprises a chemical to prevent Ostwald ripening, wherein the chemical to prevent Ostwald ripening is a salt. The high internal phase emulsion further comprises an oxidizing agent is in a quantity that maintains the high internal phase emulsion as an emulsion and is insufficient to initiate extensive free radical cross-linking of the biodegradable polymeric material when stored at ambient temperatures. The high internal phase emulsion further comprises an oxidizing agent is in a quantity that maintains the high internal phase emulsion as an emulsion and is insufficient to initiate extensive free radical cross-linking of the biodegradable polymeric material when stored at less than ambient temperatures. The high internal phase emulsion further comprises water. The water is generally in a volume of at least about 75% of the internal phase emulsion by volume. The high internal phase emulsion is stable as an emulsion without undergoing further polymerization when stored at a sub-ambient temperature. The octanol-water partition coefficient of the biodegradable polymeric material may be between about 2 and about 4. The viscosity of the biodegradable polymeric material may be near the viscosity of water. The oxidizing agent may be a free radical oxidizing initiator. The hydrophilic-lipophilic balance of the emulsifier may be in a range of between about 3 and about 5.

A high internal phase emulsion when formed may comprise a biodegradable polymeric material. The biodegradable polymeric material comprising: at least one end group selected from an acrylate and a methacrylate; and one or more linkages selected from an anhydride and an ester. The biodegradable polymeric material generally has an octanol-water partition coefficient of between about 2 and about 8 and a viscosity of between about 0.08 cP and about 1000 cP. The biodegradable polymeric material is stabilized with a quantity of an emulsifier lacking hydrogen bond donors in its hydrophilic head region while having a hydrophilic-lipophilic balance in a range of between about 2 and about 9. The high internal phase emulsion further comprises a chemical to prevent Ostwald ripening, wherein the chemical to prevent Ostwald ripening is in an aqueous phase. The high internal phase emulsion further comprises a reducing agent. The high internal phase emulsion further comprises water The water may be in a volume of at least about 75% of the emulsion. The high internal phase emulsion is generally stable as an emulsion without undergoing further polymerization when stored, for example at a sub-ambient temperature. The high internal phase emulsion is generally stable as an emulsion without undergoing further polymerization when stored at an ambient temperature. The octanol-water partition coefficient of the biodegradable polymeric material may be between about 2 and about 4. The viscosity of the biodegradable polymeric material may be near the viscosity of water. The reducing agent may be selected from one or more of a hydrocarbon, metal ion, vitamin, and bioactive agent. The hydrophilic-lipophilic balance of the emulsifier in a range of between about 3 and about 5.

A method of making a high internal phase emulsion may comprise combining by mechanical dispersion a first high internal phase emulsion with a second high internal phase emulsion. The first high internal phase emulsion generally comprises an oxidizing agent in a quantity insufficient to initiate extensive cross-linking in the first high internal phase emulsion when stored, for example at sub-ambient temperatures. The second high internal phase generally comprises a reducing agent.

A method of making a body using a high internal phase emulsion composition may comprise combining by mechanical dispersion a first high internal phase emulsion containing an oxidizing initiator with a second high internal phase emulsion containing a reducing agent. The method may further comprise allowing the first high internal phase emulsion and the second high internal phase emulsion to undergo a redox reaction. Upon combining the first and second emulsion a foam body is formed. The foam body generally has a porosity at or greater than 75%. In some embodiments, a rate of combining has an effect on an average pore diameter in the foam body. In some embodiments, the foam body is injectable into any of a form, a mold, and in situ, such that the foam body takes a shape of said form, said mold, or may be formed in situ. In some embodiments, the foam body has a pore size in any size or range of sizes from between about 1 micron and 300 microns. In some embodiments, the foam body cures between about 10 seconds and about 100 minutes.

A high internal phase emulsion composition is formed, the composition comprising a first high internal phase emulsion comprising at least a first biodegradable polymeric material, an oxidizing agent and water. The oxidizing agent is generally in a quantity that maintains the first high internal phase emulsion as an emulsion and delays cross-linking of the first high internal phase emulsion. The oxidizing agent is generally in a quantity that maintains the first high internal phase emulsion as an emulsion and delays cross-linking of the first high internal phase emulsion when at an ambient temperature. The water is in a volume that is at least about 75% of the first high internal phase emulsion by volume. The high internal phase emulsion composition further comprises a second high internal phase emulsion comprising at least a second biodegradable polymeric material, a reducing agent and water. The water is in a volume that is at least about 75% of the second high internal phase emulsion by volume. The reducing agent may be in a quantity that maintains the second high internal phase emulsion as an emulsion and delays cross-linking of the second high internal phase emulsion when at an ambient temperature. The high internal phase emulsion composition may further comprise one or more a bioactive component and a cell in an aqueous phase of any or both the first high internal phase emulsion and the second high internal phase emulsion. The high internal phase emulsion may further comprise a bioactive component in an organic phase of any or both the first high internal phase emulsion and the second high internal phase emulsion. The first high internal phase emulsion and the second high internal phase emulsion may be stored separately at a sub ambient temperature prior to use. The first biodegradable polymeric material and the second biodegradable polymeric material may be the same, or may each contain at least one end group selected from an acrylate and a methacrylate and one or more linkages selected from an anhydride and an ester. In some embodiments, the end groups and linkages in each biodegradable polymeric material may be similar or the same.

### BRIEF DESCRIPTION OF THE DRAWINGS

Various embodiments will be explained in more detail with reference to the drawings in which:

FIG. 1A depicts a representative cross section of a water-in-oil high internal phase emulsion;

FIG. 1B depicts a close-up of components in a boxed area of FIG. 1A;

FIG. 1C depicts schematic of a system and method described herein;

FIG. 2 depicts a representative method described herein;

FIG. 3 depicts another representative method described herein;

FIG. 4 depicts still another representative method described herein;

FIG. 5 depicts a general structure of a propylene fumarate dimethacrylate (PFDMA) macromer;

FIGS. 6A-6C depict the porous structure of polyHIPEs prepared using the propylene fumarate dimethacrylate macromer of FIG. 5 with 0.5 wt. % (FIG. 6A) or 1 wt. % (FIG. 6B) or 5 wt. % (FIG. 6C) of a redox pairing;

FIG. 7 depicts a general structure of an ethylene glycol dimethacrylate (EGDMA) macromer;

FIGS. 8A-8C depict the porous structure of polyHIPEs prepared using the ethylene glycol dimethacrylate macromer of FIG. 6 with 0.5 wt. % (FIG. 7A) or 1 wt. % (FIG. 7B) or 5 wt. % (FIG. 7C) of a redox pairing;

FIG. 9 depicts general structure of a butane diol dimethacrylate (BDMA) macromer;

FIGS. 10A-10C depict the porous structure of polyHIPEs prepared using the butane diol dimethacrylate macromer of FIG. 8 with either 0.5 wt. % (FIG. 9A) or 1 wt. % (FIG. 9B) or 5 wt. % (FIG. 9C) of a redox pairing;

FIG. 11 depicts general structure of a methacrylate anhydride macromer;

FIGS. 12A-12C depict the porous structure of polyHIPEs prepared using a methacrylic anhydride macromer with either 0.5 wt. % (FIG. 11A) or 1 wt. % (FIG. 11B) or 5 wt. % (FIG. 11C) of a redox pairing;

FIGS. 13A-13F depict storage and loss moduli during EGDMA (FIG. 13A), BDMA (FIG. 13C), and PFDMA (FIG. 13E) polyHIPE polymerization and work and set times for EGDMA (FIG. 13B), BDMA (FIG. 13D), and PFDMA (FIG. 13F) polyHIPEs at 37° C. with 1.0 TMA:1.0 BPO ratio;

FIG. 14A-I depict representative scanning electron micrographs (SEMs) of pore architecture for EGDMA polyHIPEs (14A-C), BDMA polyHIPEs (14D-F), and PFDMA polyHIPEs (14G-I);

FIGS. 15A-D depicts SEMs of PFDMA polyHIPEs after storing at 4° C. before polymerization, storage was compared to control (0 days, FIG. 15A), and included 1 month (FIG. 15B), 3 months (FIG. 15C) and 6 months FIG. 15D);

FIG. 16A-B depict the effect of initiator concentration on compressive modulus (FIG. 16A) and strength (FIG. 16B) for each material;

FIGS. 17A-C depict representative compressive loading curves for each material having redox initiator concentrations of either 0.5 wt. % (FIG. 17A), 1.0 wt. % (FIG. 17B, or 5.0 wt. % (FIG. 17C);

FIGS. 18A-B depict the effect of incubation for 1 and 14 days at 37° C. on PFDMA polyHIPEs showing compressive modulus (FIG. 18A) and compressive strength (FIG. 18B);

FIGS. 19A-C depict the effect of increasing TMA:BPO ratio in EGDMA polyHIPEs on: work and set times (FIG. 19A), compressive modulus (FIG. 19B), and compressive strength (FIG. 19C);

FIG. 20 depicts hMSC viability after 24 hour incubation with diluted extracts (1X, 10X, and 100X) from 10 minute media incubations with each macromer;

FIG. 21A depicts viability of human mesenchymal stem cells (hMSCs) after 24 hours of direct seeding as shown in micrographs of sections of polyHIPEs formed with 1% redox agents either using BDMA (top), EGDMA (middle), or PFDMA (bottom), which illustrate live cells (bright or light more elongated shapes, in green) and dead cells (dark circles, in red);

FIG. 21B shows viability of cells at 3 hour and 24 hour time points (n=20);

FIG. 22A depicts an actual representative double barrel syringe of a type described herein;

FIG. 22B illustrates a schematic for encapsulating hMSCs in an injectable HIPE using a double barrel syringe with mixing head setup;

FIG. 23 illustrates a micrograph of 3 hr hMSC viability post encapsulation in a BDMA HIPE initiated with 0.5 wt. % BPO/2.5 wt. % ferrocene, in which there were only a few dead cells (more punctate larger and darker circles, shown by arrows) surrounded by clusters of live cells (lighter, amorphous shapes or masses); and

FIGS. 24A-B depict representative pore architecture differences between a control BDMA polyHIPE (FIG. 24A) and BDMA polyHIPE in which 0.3 million cells/ml HIPE were encapsulated therein (FIG. 24B).

### DESCRIPTION

Although making and using various embodiments are discussed in detail below, it should be appreciated that as described herein are provided many inventive concepts that may be embodied in a wide variety of contexts. Embodiments discussed herein are merely representative and do not limit the scope of the invention.

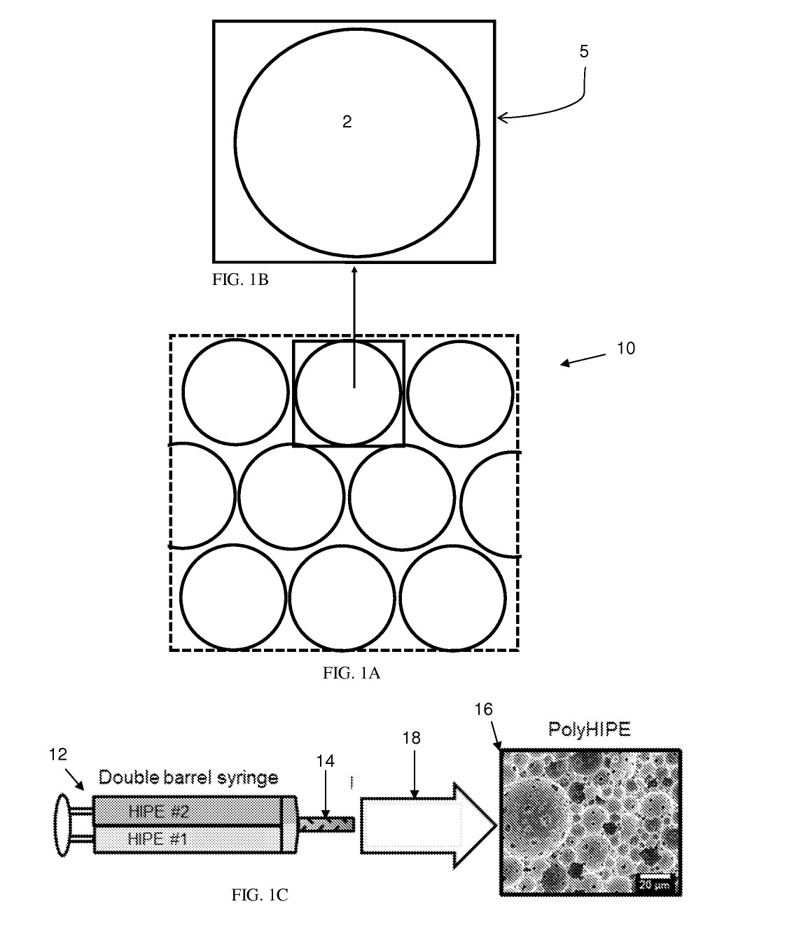

High internal phase emulsions (HIPEs) are three-dimensional polymer networks characterized herein as water-in-oil emulsion systems 10 having an internal phase volume fraction 2 comprising essentially water (which may further comprise water soluble constituents and/or bioactive molecules, cells, etc.) making up 74% or more of the total emulsion volume, thus, having only a minor, continuous phase consisting of stabilized macromers 5 (e.g., polymeric materials that are functionalized and capable of undergoing further polymerization) (FIGS. 1A and 1B). The phases are also depicted in FIG. 1B.

Fabrication of biodegradable HIPEs and polyHIPEs and their compositions have been described in U.S. patent application Ser. No. 13/651,362 (the entirety of which is incorporated herein by reference). Improvements to said biodegradable HIPEs and polyHIPEs and methods of making are now described. The improvements include controlling one or more of the following: free radical production, time to cure, average pore diameter, overall porosity and morphology, hardness, and various combinations thereof.

As described herein are various biodegradable HIPE formulations stable when prepared as an emulsion without polymerizing, though still containing a polymerizing initiator.

Additionally, as described herein are various biodegradable HIPE formulations, and biodegradable polyHIPE formulations provided therefrom, containing a reduced amount of a polymerization initiator (e.g., free radical initiator) as compared to the amounts provided in formulations described in U.S. patent application Ser. No. 13/651,362 or other comparative biodegradable HIPE formulations, and biodegradable polyHIPE formulations provided therefrom.

Also as described herein are various biodegradable polyHIPE formulations that, though containing a reduced amount of a polymerization initiator (e.g., free radical initiator), will, when formed, polymerize faster, having an increased time to cure as compared with alternative polyHIPE formulations that contain a higher amount of polymerization initiator (e.g., free radical initiator). The polyHIPEs described herein provide a number of advantages: they are injectable, cure quickly and in situ, cure in a solvent free environment and, due to their low cure temperatures permit adequate flow site specifically followed by formation into rigid bodies or foam-like bodies, all at a physiologic temperature.

The injectable polyHIPEs described herein may cure in a same time period as PMMA but with the advantages that the polyHIPEs will stabilize a bone defect and may also be loaded with cells prior to injection to provide a temporary matrix that supports tissue regeneration. In addition, polyHIPE grafts described herein remain stable in storage for a much longer period of time, by being capable of storage for up to and more than 6 months, and will still cure rapidly after injection, meaning the described polyHIPE materials are more suitable as an off-the-shelf graft for both emergency and/or scheduled procedures.

The formulations described herein are capable of creating tunable, biodegradable polyHIPEs not previously described yet still capable of polymerizing and curing under physiologic conditions. The polyHIPE formulations described herein have a reduced time to cure (cure faster) once formed. The polyHIPE formulations described herein are suitable for various in vivo, in situ and ex vivo applications that can take advantage of a highly porous polymer. The polymer chemistry chosen for the polyHIPE formulation may be further tuned to alter and manipulate structural hardness (e.g., compressive strength) when so desired.

The HIPEs as described herein are biodegradable and, as fabricated, can remain stable as an emulsion, thereby capable of delaying or suspending polymerization. Such emulsions may be stored for extended periods of time (months, years) prior to use. The HIPEs are generally prepared from a biodegradable polymeric material comprising a functionalized macromer capable of undergoing further polymerization.

The macromer used for the HIPEs described herein is either an ester based monomer, macromer, and/or polymer or an anhydride based monomer, macromer, and/or polymer and will have at least one reactive end group, which is biodegradable, a suitable hydrophobicity (determined by an octanol-water partition coefficient [LogP]) and viscosity for emulsion in water, and may be polymerized and/or cured at or near physiologic conditions. In some embodiments, the macromer itself may be prepared via a two-step reaction, such as that described in U.S. patent application Ser. No. 13/651,362, which includes: (i) backbone synthesis, and (ii) functionalization.

The at least one reactive end group of the macromer described herein is one that crosslinks at a thermal temperature or a lower temperature (e.g., ambient temperature, physiologic temperature) and of a low molecular weight that maintains a low viscosity for the macromer, hence acting to replace and no longer requiring addition of a toxic diluent. The at least one reactive end group has at least one unsaturated (double) bond for undergoing free radical cross linking. The carbon-carbon double bond in the at least one reactive end group allows thermal decomposition to occur in the presence of the redox initiators, to be described further. In one or more embodiments, the at least one reactive end group generally includes an acrylate end group or a methacrylate end group.

The hydrophobicity of the macromer described herein is defined by a LogP at about or greater than 2. It may also be defined by a LogP from between about 2 and about 8. It may also be defined by a LogP from between about 2 and about 4. A host of suitable macromers may be identified using available tools, including online services, such as one provided by Molinspiration Cheminformatics. For example, Molinspiration Cheminformatics provides model predictions of the LogP for more than 12,000 molecules or compounds, generally calculated from the sum of non-overlapping molecular fragments after fitting calculated LogP with experimental LogP values.

The LogP value of various representative compounds suitable under the definition provided herein are provided in TABLE 1, including PFDMA, which is propylene fumarate dimethacrylate; BDMA, which is butane diol dimethacrylate; EGDMA, which is ethylene glycol dimethacrylate; and MA, which is methacrylic anhydride. These suitable and representative macromers are compared to ones that are already used by alternative methods to prepare a stable HIPE, such as styrene and divinyl benzene.

TABLE 1
Estimated octanol-water partition coefficients

| Molecule | LogP |
|---|---|
| styrene | 2.8 |
| divinyl benzene | 3.6 |
| PFDA | 2.3 |
| PFDMA | 3.4 |
| BDMA | 3.0 |
| EGDMA | 2.2 |
| MA | 2.4 |

The viscosity of the macromer described herein is defined as being in a range between about 0.08 cP and about 1000 cP (at an ambient temperature). In some embodiments, the initial viscosity is near that of water, which is near 1 cP (at about ambient temperature or about 20 degrees C.). In some embodiment, the viscosity is not greater than 0.150 cP (at an ambient temperature).

The macromer as described herein may also be characterized as a biodegradable polymeric material having one or more ester linkages or a biodegradable polymeric material having one or more anhydride linkages. Exemplary embodiments include but are not limited to a biodegradable fumarate based polymer having one or more ester linkages (e.g., propylene fumarate dimethacrylate [PFDMA]), a biodegradable glycol based polymer having one or more ester linkages (e.g., ethylene glycol dimethacrylate [EGDMA]), a hydroxy (e.g., diol) based polymer having one or more ester linkages (e.g., butane diol dimethacrylate [BDMA]) and a biodegradable acrylic based polymer having one or more anhydride linkages (e.g., methacrylic anhydride [MA]).

The macromer when used for the HIPEs described is stabilized in the continuous phase by an emulsifier. The emulsifier may be an amphiphilic surfactant having a polar, water-soluble head group attached to a nonpolar, water-insoluble hydrocarbon chain. The emulsifier is selected as one lacking hydrogen bond donor sites (donors) in its hydrophilic, polar head region. The emulsifier has, some embodiments, a hydrophilic-lipophilic balance (HLB) in a range of between about 2 and about 9. In some embodiments, the emulsifier has a hydrophilic-lipophilic balance (HLB) in a range of between about 3 and about 5.

In the embodiments described, more than one independently prepared HIPE is used for fabrication of a polyHIPE. Each HIPE is a high internal phase resin emulsion formed as a water-in-oil emulsion comprising a stabilized biodegradable polymeric material in its continuous (oil) phase and at least a redox polymerizing initiator; the redox polymerizing initiator may be in the dispersed (water) phase (when aqueous) or the continuous phase (when non-aqueous). The redox initiator is, in a first high internal phase resin emulsion, an oxidizing initiator (oxidant) and is, in second high internal phase resin emulsion, a reducing initiator (reductant). Thus, a high internal phase resin emulsion will either have an oxidizing initiator or a reducing agent. In one or more embodiments, decomposition is not initiated in either the first high internal phase resin emulsion or the second high internal phase resin emulsion when prepared as described herein. In some embodiments, decomposition is delayed in either the first high internal phase resin emulsion or the second high internal phase resin emulsion when prepared as described herein.

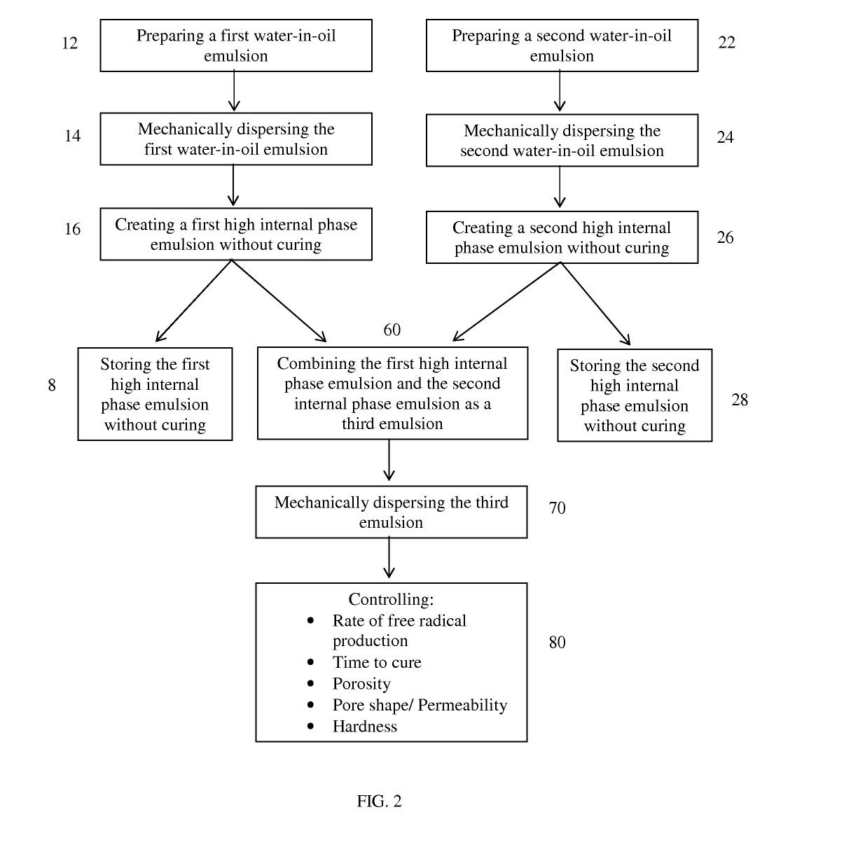

In a first high internal phase resin emulsion, components will include a stabilized macromer, an oxidizing initiator, a chemical preventing Ostwald ripening and water. Water will be in an amount of about or greater than 75% (v/v). The at least one chemical that prevents Ostwald ripening may be a salt or an electrolyte. Only a small amount of the chemical preventing Ostwald ripening is typically required, generally about 1 to about 5% (v/v). The oxidizing initiator is selected as one that generates free radicals by thermal or ambient decomposition. The oxidizing initiator is a free radical oxidizing agent, such as, but not limited to a peroxide, a persulfate, or an azo compound initiator. Representative examples include but are not limited to azobis-isobutyronitrile (AIBN) and benzoyl peroxide (BPO). The organic and soluble free radical initiators are useful for polyHIPEs introduced in vivo because these do not appear to leach components from the scaffold that effect cell growth and viability (data not shown). The oxidizing initiator is either a non-aqueous free radical oxidizing agent or an aqueous free radical oxidizing agent. The quantity of oxidizing initiator in the first HIPE composition is considered to be low, in an amount that is insufficient to initiate extensive free radical cross-linking of a macromer chain (of unsaturated double bonds of the one or more end groups). The amount will vary from between about 1 wt. % to about 10 wt. % or about 15 wt. % (based on the total weight in the organic phase) but will be less than what would be required were the high internal phase resin emulsion to undergo polymerization to form a polyHIPE. The reduced concentration of the oxidizing initiator described herein, not found in previous HIPES, ensures stability of the first high internal phase resin emulsion and delays and/or prevents polymerization at a physiologic or ambient temperature. As such, these HIPEs may be stored without fully polymerizing. The HIPEs described herein are, thus, suitably stable as an emulsion at a physiologic or an ambient temperature without setting (curing). These HIPEs may be stored at a temperature that is a physiologic or ambient or may be frozen for later use as depicted in box 18, FIG. 2.

This first high internal phase resin emulsion is prepared as illustrated in FIG. 2, boxes 12, 14, and 16, in which the stabilized macromer is in the continuous phase and the remaining components are generally in the dispersed (aqueous) phase. When the initiator is a non-aqueous initiator, the initiator will be in the continuous phase. Once the macromer and its stabilizer (emulsifier) are thoroughly mixed, additional components are added, typically in an aqueous phase. Components, in addition, to the initiator and water include at least one or more of a chemical that prevents Ostwald ripening. One or more further biologic constituents may be included in the aqueous phase as desired for a controlled supply or delivery of said biologic constituents. These biologic constituents may include physiologic components or their synthetic equivalents, such as media, serum, cells, growth factors or other bioactive factors, and proteins, and the like. A biologic constituent may also have a bioactive modifier, one that enables the biologic constituent to behave in a biologic or physiologic manner. The bioactive modifier is typically small (about 5-200 nanometers) and often includes a hydrophobic component or moiety. An example of a bioactive modifier is but is not limited to an inorganic nanoparticle. The inorganic nanoparticle may be further linked to a hydrophobic component or fatty acid. Another example of a bioactive modifier is but is not limited to an amphiphilic molecule having a cell-adhesion or adhesive-like moiety (e.g., fatty acid conjugated to cell-adhesion molecule, peptide or protein).

In an example of a first HIPE, a macromer (comprising 1 g propylene fumarate dimethacrylate [PFDMA]) was initially blended in a mixer (a dual asymmetric centrifugal mixer) with a stabilizer lacking hydrogen bond donor sites at its polar head region (20 wt. % polyglycerol polyricinoleate [PGPR]), to form a stabilized macromer. The stabilized macromer was emulsified with a non-aqueous initiator (0.5 wt. % benzoyl peroxide [BPO]) and a chemical that prevents Ostwald ripening (1% (v/v) calcium chloride) in deionized water (3 g) by an additional blending in the dual asymmetric centrifugal mixer (500 rpm). The emulsion prepared was a high internal phase emulsion. This emulsion did not set at physiologic temperature (e.g., about 37° C.) when evaluated for 72 hours.

In a second high internal phase resin emulsion, components will include a stabilized macromer, a reducing agent, a chemical preventing Ostwald ripening and water. Water will be in an amount of about or greater than 75% (v/v). The at least one chemical that prevents Ostwald ripening may be a salt or an electrolyte. Only a small amount of the chemical preventing Ostwald ripening is typically required, generally about 1 to about 5% (v/v). The reducing agent may be a hydrocarbon, inorganic compound, biological agent, vitamins, metal ion, and the like. The quantity of the reducing agent is in an amount that is insufficient to initiate free radical cross-linking of a macromer chain (of unsaturated double bonds of the one or more end groups). The amount will generally vary from between about 0.5 wt. % to about 10 wt. %. The low concentration of the reducing agent ensures stability of the second high internal phase resin emulsion and delays and/or prevents its polymerization at a physiologic or ambient temperature. As such, these HIPEs may be stored without fully polymerizing and are, thus, suitably stable as an emulsion at a physiologic or ambient temperature without setting (curing). These HIPEs may be stored at a temperature that is a physiologic or ambient or may be frozen for later use as depicted in box 28, FIG. 2.

This second high internal phase resin emulsion is prepared as illustrated in FIG. 2, boxes 22, 24, and 26, in which the stabilized macromer is in the continuous phase and all or a majority of the remaining components are in the dispersed (aqueous) phase. This preparation is similar to the preparation of the first high internal phase emulsion. The primary difference being that the oxidizing agent (in the first high internal phase resin emulsion) has been replaced by a reducing agent in the second high internal phase resin emulsion. Thus, once the macromer and its stabilizer (emulsifier) are thoroughly mixed, additional components are added, typically in an aqueous phase. Components, in addition, to the reducing agent and water include at least one or more of a chemical that prevents Ostwald ripening. One or more further biologic or other type of constituents may be included in the aqueous phase as desired for a controlled supply or delivery of said constituent. These constituents may be physiologic (or biologically derived, e.g., media, serum, cells, growth factors, bioactive factors, proteins, and the like), or their synthetic equivalents, or have some biologic activity (drug, chemoactive agent). A biologic constituent may also have a modifier, one that enables the biologic constituent to behave in a biologic or physiologic manner. The bioactive modifier is typically small (about 5-200 nanometers) and often includes a hydrophobic component or moiety. An example of a bioactive modifier is but is not limited to an inorganic nanoparticle. The inorganic nanoparticle may be further linked to a hydrophobic component or fatty acid. Another example of a bioactive modifier is but is not limited to an amphiphilic molecule having a cell-adhesion or adhesive-like moiety (e.g., fatty acid conjugated to cell-adhesion molecule, peptide or protein).

The two independently prepared first and second high internal phase resin emulsions are combined to fabricate a polyHIPE via a redox reaction (FIG. 2, box 60 and 70). The polyHIPE when formed will generally be biodegradable. The polyHIPE, as fabricated, is fast curing, providing a three dimensional architecture with an open pore morphology. By fabricating under the described conditions, there is the ability to control one more of the following: free radical production; time to cure; porosity (e.g., from between about 75% to about-99%), average pore size (e.g., from between about 2 μm to about 50 μm or from about 15 to about 40 μm), compressive modulus (e.g., from between about 2 MPa to about 50 MPa), and strength (e.g., from between about 1 to about 10 MPa), as depicted in box 80, FIG. 2

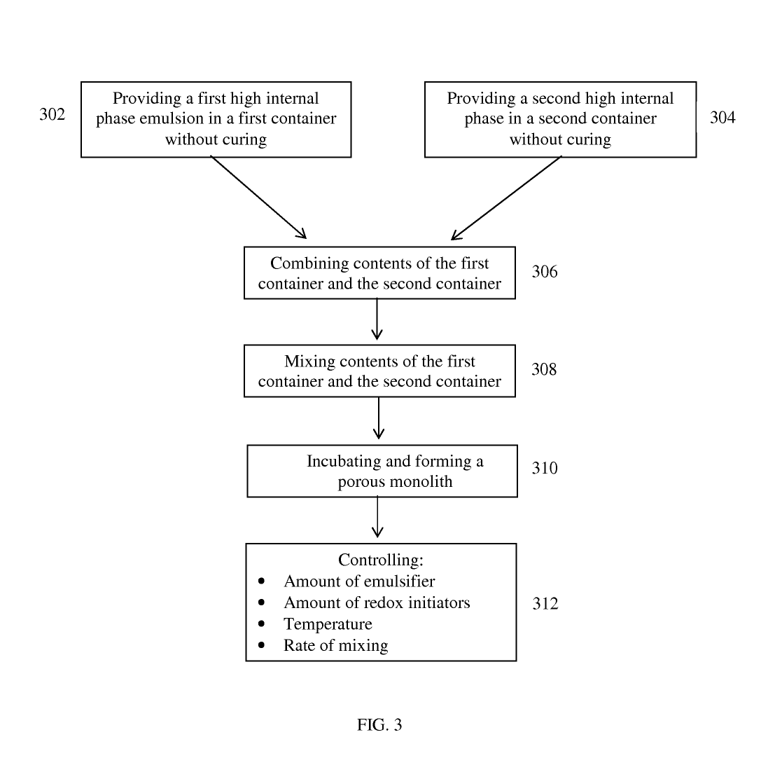

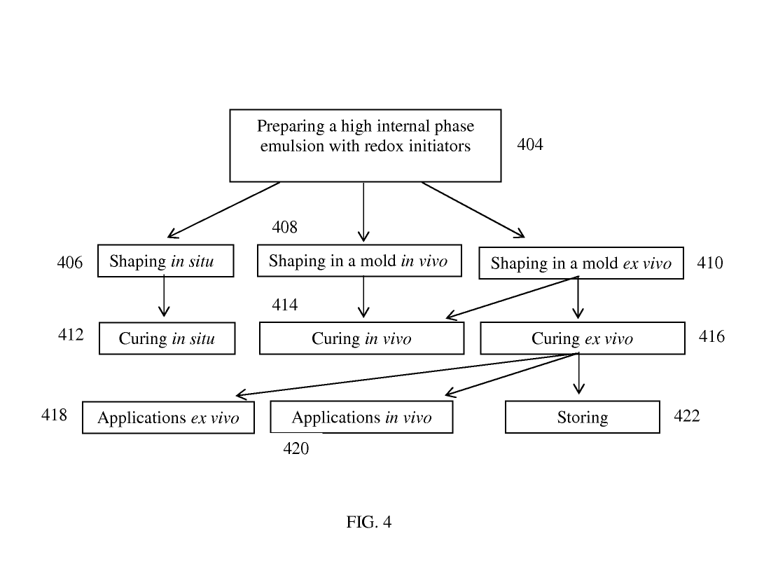

The making of representative polyHIPEs is outlined in FIGS. 3 and 4. A first HIPE in a first container (box 302, FIG. 3) and a second HIPE in a second container (box 304, FIG. 3) are combined by a suitable means for combining (box 306, FIG. 3) and thereafter blended to form a final emulsion (box 308, FIG. 3). The means for combining includes any available means to combine such emulsions. The means for combining may also provide the means for blending the first and second emulsion. Representative examples include but are not limited to extrusion via a double barreled syringe or via other co-mixing or co-extruding methods. The final emulsion is suitable for injection, having a mayonnaise-like viscosity, thereby capable of holding a shape yet also capable of intermixing, at an interface, with one or more physiologic components when introduced in vivo or in situ. The final emulsion once combined is allowed to react (box 310, FIG. 3). The inclusion of the reducing agent in the presence of the lower concentration of the initiating agent increases the reaction kinetics of the final emulsion described herein, a reaction that is much higher as compared to alternative HIPEs that have only an initiator for initiating polymerization, even though the initiator (in alternative HIPEs) is in a higher concentration than what is found in the first high internal phase resin emulsion described herein. The increased reaction kinetics translated into a significant decrease in time to cure of the final emulsion. For example, the time to cure of a polyHIPE described herein may be in a few seconds to several minutes and less than one hour, as compared to a polyHIPE prepared with the same macromer and initiator but lacking a reducing agent, which will exhibit a time to cure of one or more hours, and generally at least two hours or more. The reaction described herein will run to completion, such that decomposition of the redox initiators promotes polymerization and crosslinking of unsaturated bonds along macromer end groups of the first and second HIPEs. The rate of decomposition of the oxidizing initiator is increased in the presence of the reducing agent. With addition of an extra, non-thermal initiator (e.g., light-activated initiator; electrochemical initiator) in at least one of the first HIPE or second HIPE. This may be desired in certain instances.

Decomposition and polymerization of the final emulsion provides a three-dimensional architecture that is in the form of a foam body. The foam body is a porous monolith. Decomposition may occur at a thermal temperature, at a physiologic temperature (e.g., temperature at about or less than about 40° centigrade) or an ambient temperature. The extent of polymerization is generally dependent on the amount of the redox initiators and the reaction temperature (box 312, FIG. 3). It is found that increasing the production of free radicals increases the rate of polymerization (and cross-linking) and reduces the cure time or set time of the foam body. Thus, described herein is the ability to adjust the relative ratio of redox initiators to directly affect the time to cure of the foam body. The reactivity of the unsaturated double bonds on the reactive end groups of the macromer is amenable to being modified in order to also effect time to cure (rate of polymerization) of polyHIPEs described herein. It is found that increasing the reactivity or number of reactive sites on the reactive end groups decreases time to cure.

The porosity and pore size is generally dependent on the rate of mixing when forming the emulsion (box 312, FIG. 3). It is found that decreasing the rate of mixing in the mixing chamber increases the average pore diameter in the foam body. The pore size is also dependent on the temperature for setting (polymerizing) the final emulsion (box 312, FIG. 3). It is found that increasing the temperature for making and/or curing the foam body increases the average pore cross-sectional diameter in the foam body and the final product when cured. The pore size is also dependent on the overall amount of emulsifier in the final emulsion (box 312, FIG. 3). It is also found that decreasing the overall emulsifier concentration increases, on average, the pore cross-sectional diameter in the foam body and the final product when cured (box 312, FIG. 3).

In an example, a polyHIPE was formed from a PFDMA macromer contained in the first HIPE and in the second HIPE, in which the final formed product had an open pore morphology, many of which were, on average, approximately 20 micrometers in diameter, with an about 75% overall porosity and an average compressive modulus of about 30 MPa and strength of about 5 MPa. Complete polymerization was obtained within a few seconds after forming the emulsion at a physiologic temperature (between 37 and 40° C.). In general, a polyHIPE when formed will exhibit at least about 75% or greater porosity. Additionally, a polyHIPE described herein when formed may have an average pore size ranging at least from about 4 μm to at least about 29 μm.

As illustrated in FIG. 4, forming a polyHIPE composition which contains redox initiators (box 404) using methods and compositions described above provide an emulsion that may be directly introduced in vivo, in situ or ex vivo. The emulsion may be shaped at the site of introduction, via injection in situ (box 406) or in a mold in vivo (box 408), or in a mold ex vivo (box 410). As such, polyHIPEs described herein may be cured or allowed to set in situ (box 412) or in vivo (box 414), or ex vivo (box 416). By introducing and allowing said polyHIPEs to cure in situ, the fully formed product when fully cured will be able to adapt to otherwise problematic or irregular geometries. Thus, the injectable form will prevent costly and time-consuming design molds and post-fabrication modifications. When curing ex vivo, such as for later use (either for physiologic or non-physiologic use), the polyHIPE may be stored (box 422) or may be used for an ex vivo application (box 418) or an in vivo application (box 420). The interconnected yet porous nature of polyHIPEs described herein provide a stable structure for promoting cellular ingrowth and may be prepared to contain biologic constituents that promote cellular connectivity and/or proliferation as well as to deliver these biologic constituents site specifically.

The ability to synthesize a fully biodegradable polyHIPE without a toxic diluent that can also rapidly cure at a selected cure rate by controlling the amount of emulsifier, amount or redox initiators, rate of mixing (for emulsification) and/or polymerization temperature is an important adaptation of emulsion templating that is described herein. The improved polyHIPEs described herein are suitable for injection and/or for molding. The improved polyHIPEs offer sufficient strength to be structural and supportive. Said HIPEs and polyHIPEs may be stored for weeks, months or at least a year (i.e. frozen and later thawed) without notable changes in architecture or strength or, for HIPEs, without notable changes in their ability to polymerize when combined as described herein (data not shown). The polyHIPEs when fully formed and cured also exhibit sufficient mechanical strength and modulus to withstand physiological loading, which is necessary to promote and restore tissue function without causing deleterious stress-shielding effects.

Another advantage of the methods of compositions described herein is that either or both initial HIPEs (the first high internal phase emulsion composition or the second high internal phase emulsion composition) may be prepared in batches, in which the type and/or concentration of emulsifier or the type and/or concentration of initiator are specifically altered. Thus any number of combinations may be introduced together to provide finely tunes polyHIPE products with selected pore size, range of pore sizes, or hierarchical pore size distributions (e.g., larger pores surrounded by smaller, monodisperse pores).

Yet, another advantage, particularly in a commercial setting, is the ability to provide a vast repertoire of polyHIPEs simply by adjusting the ratio of a first high internal phase emulsion composition to a second high internal phase emulsion composition, which will affect the rate of polymerization or time to cure without changing initial HIPE fabrication parameters. When one of the first high internal phase emulsion composition or the second high internal phase emulsion composition contains an additive or biologic constituent (either in its organic or aqueous phase), the final concentration in the polyHIPE can be readily altered by again simply changing the ratio of either or both of the starting HIPEs. Further modifications may be made to polyHIPEs prepared ex vivo by modifying the cure temperature.

Previously described PFDMA macromers have been used to fabricate polyHIPEs using a large amount of a free radical oxidizing initiator alone, including either 30 wt. % ammonium persulfate (APS) or 5 wt. % BPO; such polyHIPEs were found to take at least 2 hours to set for 1 g of PFDMA polyHIPE (Moglia R S, Holm J L, Sears N A, Wilson C J, Harrison D M, and Cosgriff-Hernandez E. Biomacromolecules 2011; 12(10):3621-3628). The resulting PFDMA macromer had a sufficiently low viscosity (about 125 cP) and hydrophobicity to permit HIPE formation. These PFDMA macromers also had a single fumarate unit with two terminal methacrylate groups. The average functionalization was calculated to be greater than 80%. In some embodiments, functionalization was calculated to be at or about 83%. The methacrylate and fumarate groups provided sites for radical crosslinking and polyHIPE fabrication.

In a first example, propylene fumarate dimethacrylate (PFDMA) polyHIPEs were prepared using a similarly prepared PFDMA macromer as described in Moglia et al. The PFDMA macromer has a general structure as depicted in FIG. 5. A first HIPE was prepared with the PFDMA macromer, 0.5 wt. % BPO, 10 wt. % PGPR, 1 wt. % calcium chloride and water. This concentration of oxidizing initiator did not promote polymerization of the first HIPE. A second HIPE was prepared with the PFDMA macromer, 0.5 wt. % 4,N,N-trimethylaniline (TMA), 10 wt. % PGPR, 1 wt. % calcium chloride and water. This concentration of reducing agent did not promote polymerization of the second HIPE. The first HIPE was placed in a first barrel of a double barreled syringe. The second HIPE was placed in a second barrel of the double barreled syringe. A mixing head was attached and the two first and second high internal phase emulsions were co-extruded through the mixing head into a mold which was placed into a water bath that was at a temperature of 37° C. The final polyHIPE as a foam reached a set point in 5 minutes.

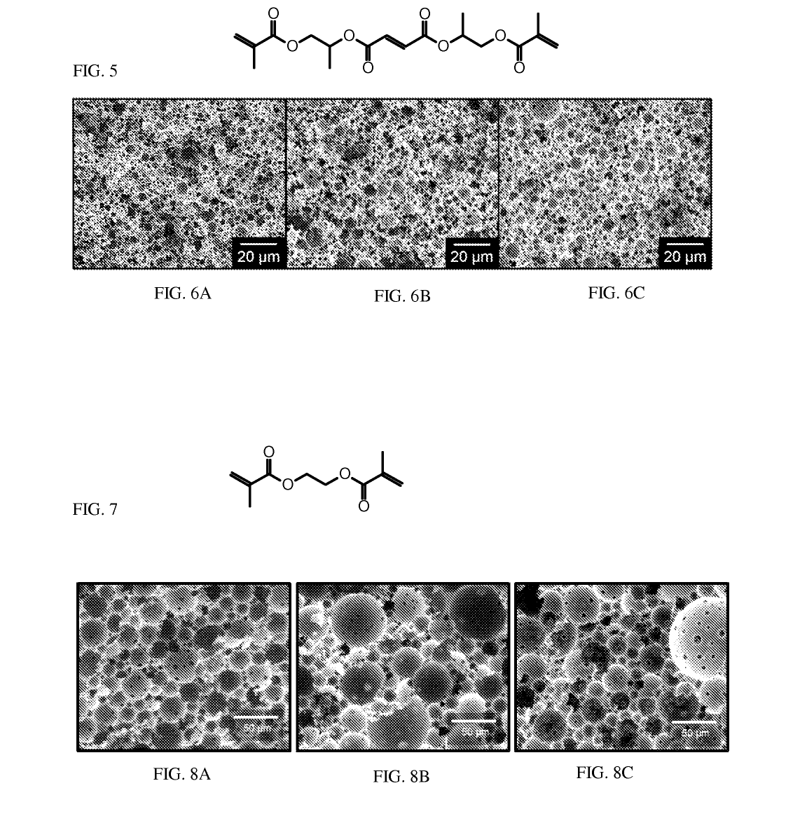

In a second example, ethylene glycol dimethacrylate (EGDMA) polyHIPEs were prepared using an EGDMA macromer having a general structure as depicted in FIG. 6. A set of first HIPEs were prepared with either the EGDMA macromer and 0.5 wt. % BPO or the EGDMA macromer and 1.0 wt. % BPO or the EGDMA macromer and 5.0 wt. % BPO. The first HIPEs all contained 10 wt. % PGPR, 1 wt. % calcium chloride and were 75% water. These first HIPES did not generally promote polymerize with the amount of oxidizing initiator used for these first HIPEs (at least for 10 hours and only when the highest amount was included). A set of second HIPEs were prepared with the EGDMA macromer and 0.5 wt. % TMA, or the EGDMA macromer and 1.0 wt. % TMA, or the EGDMA macromer and 5.0 wt. % TMA. The first and second HIPEs all contained 10 wt. % PGPR, 1 wt. % calcium chloride and were 75% water. It was found that none of the second HIPES polymerized in the presence of the reducing agent (TMA).

Combinations of the first HIPE and the second HIPE were incubated together and molded in a manner similar to what was described in the first example to form porous polyHIPEs. These redox formed polyHIPEs were evaluated for their time to cure and compared to the time to cure of the first HIPEs and the second HIPES (when uncombined, therefore containing either only an oxidizing initiator or only a reducing initiator/agent). It was found that the time to cure for the redox formed polyHIPEs could be controlled. Not only was the cure time dramatically reduced when redox initiators were used, but by altering the amount of redox initiators, the time to cure could be quickly and easily manipulated. Thus, fast curing polyHIPEs were created that could cure in less than comparative times (which were at least two hours). As described herein, cure times were less than two hours. They could be designed to cure for much less than two hours. For example, the cure times could be for less than 1 hour, or less than thirty minutes, or less than about 20 minutes to as little as 30 seconds. Some representative time to cure values for the first HIPEs (alone), the second HIPEs (alone) and the combinations are provided in TABLE 2.

TABLE 2

| | Initiator | Time to cure |
|---|---|---|
| polyHIPE | (wt. %) | (minutes) |
| first HIPE | 0.5 BPO | did not cure |
| first HIPE | 1.0 BPO | did not cure |
| first HIPE | 5.0 BPO | 600 to 1200 |
| first HIPE + | 0.5 BPO + | 20 |
| second HIPE | 0.5 TMA | |
| first HIPE + | 1.0 BPO + | 5 |
| second HIPE | 1.0 TMA | |
| first HIPE + | 5.0 BPO + | 0.5 |
| second HIPE | 5.0 TMA | |
| second HIPE | 0.5 TMA | did not cure |
| second HIPE | 1.0. TMA | did not cure |
| second HIPE | 5.0 TMA | did not cure |

The redox formed polyHIPEs (ones prepared with a combination of a first HIPE and second HIPE) were analyzed by scanning electron microscopy when fully cured. For SEM sampling, circular specimens were sectioned into quarters, fractured at the center of the quarter, sputter-coated with gold, and imaged using FE-SEM (JEOL JSM-7500F). Images at 250× were used to determine the average pore size when the pores were 25-100 μm. Higher magnification (500×, 1000×) images were utilized to determine the average pore size when the pores were less than 25 μm. Each section was imaged in a raster pattern yielding five images. Measurements were made on at least the first 10 pores along the image median to minimize user bias. Averages pore sizes for each polyHIPE composition are shown (n=150). A statistical correction was calculated to account for non-perfect spherical pores, h²=R²−r², where R is the void diameter's equatorial value, r is the diameter value measured from the micrograph, and h is the distance from the center. The average diameter values were multiplied by this correction factor resulting in a more accurate description of pore diameter. Representative SEM images are provided in which the average pore size was found to increase with increasing concentration of redox initiator pairs (see FIG. 7A with 0.5 wt. % of each redox initiator; FIG. 7B with 1.0 wt. % of each redox initiator; FIG. 7C with 5.0 wt. % of each redox initiator). The average pore diameter was about 26 μm and interconnects were about 4 μm, on average.

For mechanical testing, redox formed polyHIPEs were mechanically tested with an Instron 3300, equipped with a 1000-N load cell. Generally, three specimens were taken from each sample. The data was then averaged from three sections for each sample tested (n=9). The test specimens were cut into flat rectangular shapes (9×9×3 mm) and compressed at 50 μm/s. Calculations were generally in accordance with ASTM method D1621-04a to determine the compressive modulus. A straight edge and computer software were used to determine the linear region of the stress-strain curve by extending a line from the steepest slope of the curve to the zero-load axis. The point at which this line crossed the axis was determined to be where strain equaled zero and all data points were shifted accordingly. The elastic modulus was equal to the slope of the line in the linear region, as outlined in ASTM D1621-04a. On average, the compressive modulus was dependent on initiator concentration and macromer chemistry. A maximum compression modulus was about 46 MPa and a maximum strength was about 5 MPa.

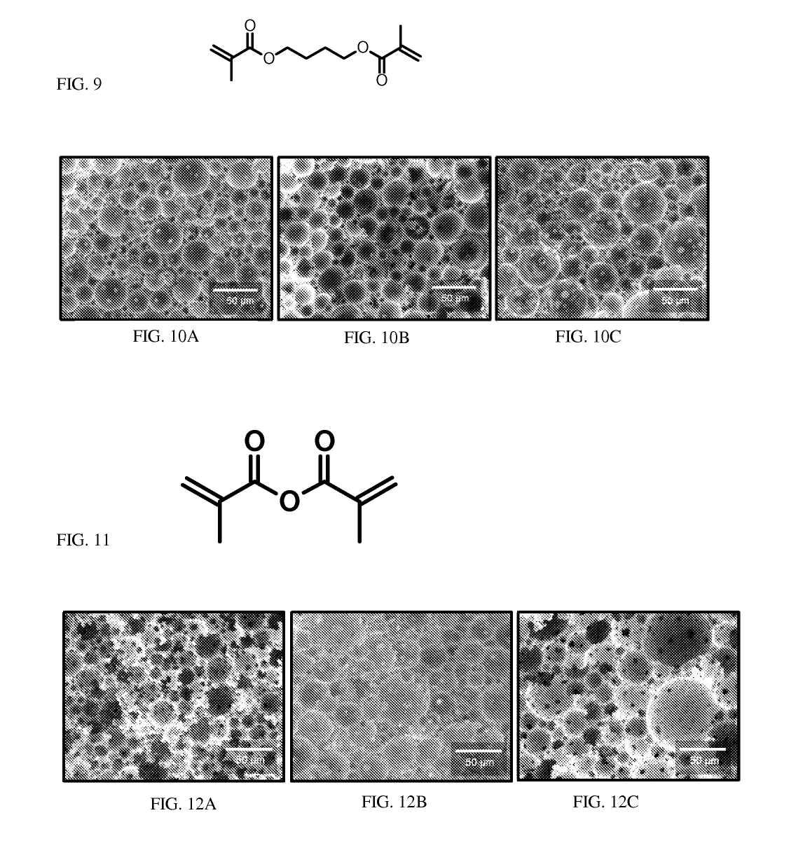

In a third example, butanediol dimethacrylate (BDMA) polyHIPEs were prepared using a BDMA macromer having a general structure as depicted in FIG. 8. A set of first HIPEs were prepared with either the BDMA macromer and 0.5 wt. % BPO or the BDMA macromer and 1.0 wt. % BPO or the BDMA macromer and 5.0 wt. % BPO. A set of second HIPEs were prepared with the BDMA macromer and 0.5 wt. % TMA, or the BDMA macromer and 1.0 wt. % TMA, or the BDMA macromer and 5.0 wt. % TMA. Various combinations of the first HIPE and the second HIPE were blended and molded in a manner similar to what was described in the first example. The time to cure was also evaluated in the redox combinations and in the first HIPEs alone and the second HIPEs alone. Only the redox-formed polyHIPEs exhibited a fast time to cure. Time to cure was also controlled by increasing the amount of redox initiators. The time to cure for a polyHIPE fabricated after combining the first HIPE with 5.0 wt. % BPO and the second HIPE with 5.0 wt. % TMA was 5 minutes. This is contrasted with a time to cure of more than 10 hours for first HIPE with 5.0 wt. % BPO. Representative SEM images of redox-formed polyHIPEs when fully cured are illustrated in FIGS. 9A to 9C, in which the redox initiator amounts were either 0.5 wt. % (FIG. 9A) or 1.0 wt. % (FIG. 9B) or 5.0 wt. % (FIG. 9C). The average pore diameter was about 20 μm and interconnects were about 4 μm, on average.

In a fourth example, methacrylic anhydride (MA) polyHIPEs were prepared using a MA macromer as depicted in FIG. 10. A set of first HIPEs were prepared with either the MA macromer and 0.5 wt. % BPO or the MA macromer and 1.0 wt. % BPO or the MA macromer and 5.0 wt. % BPO. A set of second HIPEs were prepared with either the MA macromer and 0.5 wt. % TMA or the MA macromer and 1.0 wt. % TMA or the MA macromer and 5.0 wt. % TMA. PolyHIPEs formed from combinations of the first HIPE and the second HIPE were fabricated and molded using the method described in the first example. The time to cure was faster for all the redox formed polyHIPEs. The time to cure was also controlled by adjusting the amount of the redox initiator; the fastest cure times were found with the combinations having the highest amounts of the redox initiators. This was contrasted with the failure to polymerize for any of the first HIPEs alone or the second HIPEs alone. The time to cure was 100 minutes for the redox formed polyHIPEs prepared with 0.5 wt. % BPO and 0.5 wt. % TMA. The time to cure was 7 minutes for the redox formed polyHIPEs prepared with 5.0 wt. % BPO and 5.0 wt. % TMA. Representative SEM images of redox formed polyHIPEs when fully cured are illustrated in FIGS. 11A to 11C, in which the redox initiator pair amounts were either 0.5 wt. % (FIG. 11A) or 1.0 wt. % (FIG. 11B) or 5.0 wt. % (FIG. 11C). The average pore diameter was about 30 μm and interconnects were about 5 μm, on average.

Representative systems and methods are further described below for fabrication of injectable polyHIPEs prepared from biodegradable macromers, said polyHIPEs capable of being stored for months at a time and then still able to cure rapidly in situ. For these examples, two separate but near-identical HIPEs (1 and 2) are used, as depicted in FIG. 1C. HIPE 1 has an oxidizing initiator and HIPE 2 has a reducing agent; said HIPEs are prepared as described. The use of a double-delivery system 12 keeps the components separate and unpolymerized until components in HIPE 1 and HIPE 2 are mixed together, which occurs upon injection via a static mixing head 14. polyHIPEs 16 were formed (e.g., in about 10 minutes) either in situ or in a warm water bath at 37 degrees Centigrade (arrow 18). The selection of suitable redox-paired initiators allowed for rapid polymerization at a low temperature. By carefully selecting initiator concentrations, said systems permit stable storage of uncured emulsions (e.g., HIPE 1 and HIPE 2). Further, said systems still rapidly cure after injecting into a specific location, such as a defect site.

In the below examples, three materials are prepared: ethylene glycol dimethacrylate (EGDMA), butanediol dimethacrylate (BDMA), and propylene fumarate dimethacrylate (PFDMA). The effects of redox concentration and ratio on cure time, pore architecture, and compressive modulus and strength were evaluated in relation to use in orthopedic applications, and demonstrated the applicability of fabricating the described rapid-curing polyHIPEs that have long shelf-lives and subsequent use as tissue engineered bone grafts.

PFDMA was synthesized in a two-step process. First, propylene oxide was added drop wise to a solution of fumaric acid and pyridine in 2-butanone (2.3:1.0:0.033 mol) and refluxed at 75° C. for 18 hours. Residual propylene oxide and 2-butatone were removed by distillation and the product redissolved in dichloromethane. Residual acidic byproducts and water were removed with washing, and the product dried under vacuum to yield the diester bis (1,2 hydroxypropyl) fumarate product. The diester was then endcapped with methacrylate groups using methacryloyl chloride in the presence of triethylamine. The molar ratios of the diester, methacryloyl chloride, triethylamine were 1:2.1:2.1, respectively. Hydroquinone was added to the diester to inhibit crosslinking during synthesis at a molar ratio of 0.008:1. The reaction was maintained below −10° C. to reduce undesirable side reactions and stirred vigorously under a nitrogen blanket. The macromer was neutralized overnight with 2 M potassium carbonate. Residual triethylamine and methacrylic acid were removed with an aluminum oxide column (7 Al₂O₃:1 TEA). The integration ratio of methacrylate protons to fumarate protons in the ¹H NMR spectra was used to confirm >90% functionalization for all macromers prior to polyHIPE fabrication. (300 MHz, CdCl₃) δ 1.33 (dd, 3H, CH₃), 1.92 (s, 3H, CH₃), 4.20 (m, 2H, —CH₂), 5.30 (m, 1H, —CH), 5.58 (s, 1H, —C═CH₂), 6.10 (s, 1H, —C═CH₂), 6.84 (m, 2H, —CH═CH⁻).

EGDMA and BDMA (purchased from Sigma Aldrich) were purified to remove inhibitors prior to use. The macromers were filtered through an aluminum oxide column to remove monomethyl ether hydroquinone. The purified products were stored at 4° C. under a nitrogen blanket until used for HIPE fabrication.

HIPEs were fabricated using a FlackTek Speedmixer DAC 150 FVZ-K. Briefly, a macromer was mixed with 10 wt. % PGPR 4125 and benzoyl peroxide (varied from 0.5-5.0 wt. %) prior to emulsification. A second mixture consisting of macromer, 10 wt. % PGPR, and a varied amount of trimethylaniline (TMA, 0.5-5.0 wt. %) was also combined prior to emulsification. Once both were thoroughly mixed, an aqueous solution of calcium chloride (1 wt. %) was then added to the organic phases (75% v) in 3 additions and mixed at 500 rpm for 2.5 minutes each. HIPEs were placed in double barrel syringe and the two components mixed upon injection using a static mixing head (5 mL syringe with 3 cm straight mixer, Sulzer Mixpac K-System). HIPEs were then placed in a 37° C. bath to initiate polymerization (approximately 10 minutes). (See also FIG. 1C).

PolyHIPE cure times were characterized using an Anton Paar MCR 301 rheometer. HIPEs were injected through a mixing head to facilitate redox initiation directly onto the 37° C. plate. Storage and loss moduli were measured every 15 seconds using a parallel-plate configuration with a 1 mm gap and 0.5% strain. Work time was presented as the onset of increasing storage modulus and set time was presented as the tan 6 minimum, which corresponds to storage modulus yielding.

PolyHIPEs were dried in vacuo for about 24 hours to remove water prior to characterization of pore architecture. Average pore and interconnect size of each composition was determined using scanning electron microscopy (SEM, JEOL 6500). Circular specimens from three separate polyHIPE specimens were sectioned into quarters and fractured at the center. Each specimen was coated with gold and imaged in a rastor pattern yielding five images. Pore size measurements were completed on the first ten pores that crossed the median of each 500× magnification micrograph. Average pore sizes for each polyHIPE composition were reported (n=450). A statistical correction was calculated to account for the random fracture plane through spherical voids and pores, 2/\sqrt{3}. Average diameter values were multiplied by this correction factor resulting in a more accurate pore diameter description.

PolyHIPE compressive properties were measured using an Instron 3300 equipped with a 1000-N load cell. ASTM D1621-04a was utilized to determine the compressive modulus and strength of the polyHIPEs. Each polyHIPE specimen was sectioned into three discs (approximately 15 mm diameter, 5 mm thick) using an Isomet® saw (registered with Illinois Tool Works Inc. Corporation, Glenview, Ill.) and compressed at a strain rate of 50 μm/s. The compressive modulus was calculated from the slope of the linear region after correcting for zero strain and the compressive strength was identified as the stress at the yield point or 10% strain, whichever occurred first. Reported moduli and strength data were averages of 9 specimens for each composition tested.

Gel fraction was measured gravimetrically to evaluate the extent of network formation. After curing for about 24 hours, polyHIPE samples were sectioned into about 15 mm by about 1 mm discs. Mass was recorded for each specimen after vacuum drying for 48 hours, incubating in 100× dichloromethane at 20° C. for about 48 hours, and vacuum drying again until a constant mass was achieved. The final weight divided by the initial weight was assessed as the gel fraction.

For storage analysis, uncured PFDMA HIPEs were stored at 4° C. for up to six months and sampled each month to determine the impact of storage on polyHIPE architecture and mechanical properties. After a sample was removed, it was thawed for 60 minutes then injected through a syringe and cured for 48 hours prior to characterization, as described above.

Investigation of macromer cytocompatibility was performed initially prior to seeding cells directly on polyHIPE sections. The in vitro cytocompatibility of BDMA, EGDMA, and PFDMA was assessed using a modified ISO 10993-5 extraction dilution test. All macromers were further purified by washing in deionized water at a volume ratio of 1:100 and incubated in vacuum for about 24 hours prior to the experiment. Bone marrow-derived human mesenchymal stem cells (hMSCs) were obtained as Passage 1 in a cryovial from the Center for the Preparation and Distribution of Adult Stem Cells. Cells were cultured in growth media containing 16.5% fetal bovine serum (Atlanta Biologicals), 1% L-glutamine (Life Technologies) and Minimum Essential Media α (MEM α, Life Technologies) to about 80% confluency and utilized at Passages 5 and 6. Cells were trypsinized with 0.25% Trypsin-EDTA (Life Technologies) and seeded at a density of about 40,000 cells/cm² in a 96 well plate and allowed to adhere for about 24 hours. On the following day, about 100 μl of each macromer was incubated in about 300 μl growth media supplemented with 1 vol. % penicillin-streptomycin (Life Technologies) in a 48 well plate to mimic the ratio of organic to aqueous phase in the HIPE. After about 10 minute incubation at about 37° C., 5% CO₂, the supernatant above the macromers was aspirated and diluted to about 10× and about 100× solutions. This time frame was selected based on the cure rates determined previously for these macromers which approximates the maximum extraction of unreacted macromer prior to cure. Extracted and diluted media (1, 10, and 100×) was then added to cells and cultured for an additional time, generally about 24 hours. Viability was assessed utilizing the Live/Dead® assay kit (registration with Molecular Probes Corporation, Eugene, Oreg.). The analysis used a plate reader (Tecan Infinite M200Pro) with excitation/emission wavelengths of 485/528 and 528/620 for calcein-AM and ethidium homodimer-1 dyes, respectively. Viability was normalized to cells on tissue culture polystyrene.

The viability of hMSCs directly seeded on cured polyHIPEs was assessed to illustrate cytocompatibility of quick-curing redox foams. PolyHIPEs were fabricated as stated above and sectioned into about 500 μm thick wafers using an Isomet® saw. Specimens were sterilized for about 3 hours in about 70% ethanol, subjected to a wetting ladder, washed 4 times with PBS, and incubated overnight in MEM α supplemented with 40 v/v % FBS at about 5% CO₂, 37° C. Cells were seeded at about 25,000 cells/cm² (for EGDMA and BDMA) and about 100,000 cells/cm² (for PFDMA) in growth media supplemented with 1 vol. % penicillin-streptomycin and cultured for about 3 hours and about 24 hours. Cell viability was assessed as described above. Rastor imaging (5 images per specimen) was conducted on four specimens (n=20) utilizing a fluorescent microscope (Nikon Eclipse TE2000-S). Cells were manually counted to quantify viability.

Data described and/or shown are provided as a mean±standard deviation for each composition. A Student's t-test was performed to determine any statistically significant differences between compositions. All tests were carried out at a 95% confidence interval (P<0.05).

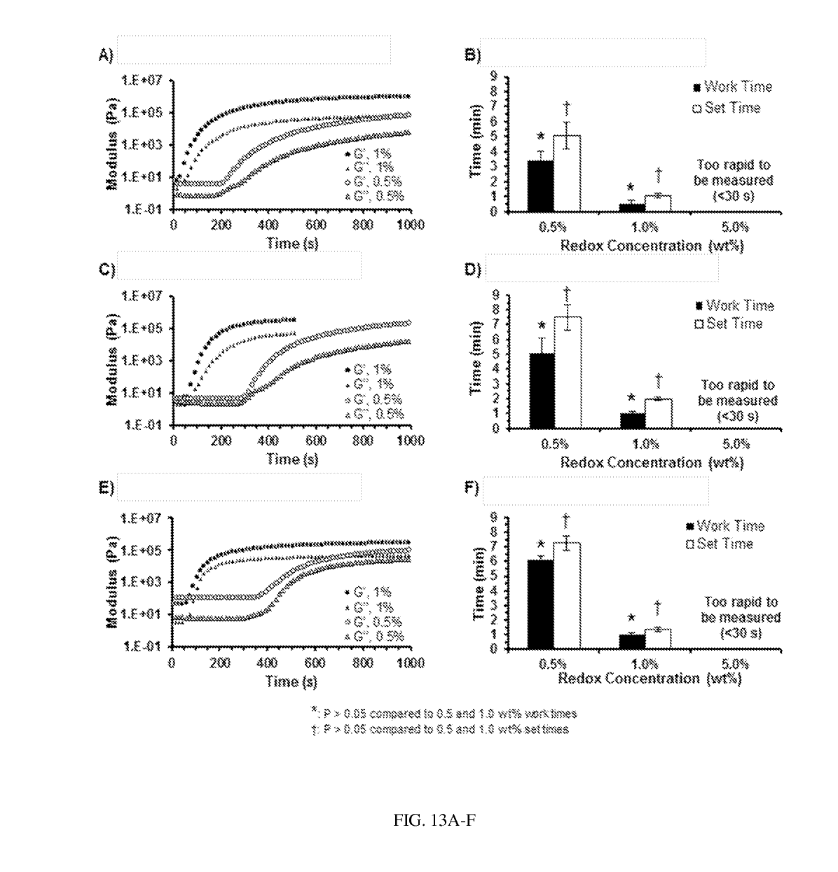

Prior to curing, all HIPEs flowed like viscous fluids but were rheologically similar to gels (E′>E″), as would be expected for HIPEs. Their moduli remained relatively constant before curing began, indicating that the emulsions were stable without significant phase separation. Work time was defined by ISO1997 as the “period of time, measured from the start of mixing, during which it is possible to manipulate a dental material without an adverse effect on its properties” and set time is accepted as the point at which a polymer network is formed. While previous cure times for PFDMA have been approximately 2 hours (with 5 wt. % BPO), the previous cure times for both EGDMA and BDMA were over 10 hours to set with thermal initiation alone. As described herein, using a reducing agent TMA in combination with BPO, it was found that both work and set time for all materials were reduced from 2+ hours to only minutes. This corresponded to an order of magnitude increase in rate as compared with thermal initiation alone. Increasing total redox initiator concentration from 0.5 wt. % to 5 wt. % was also found to decrease both work and set times for all materials, as depicted in FIGS. 13A-F. For example, for EGDMA, redox initiation with 0.5 wt. % decreased work time to 3.5 minutes and set time to 5 minutes. Increasing initiator concentration to 1 wt. % further decreased work time to 30 seconds and set time to 1 minute (FIGS. 13A-B). BDMA displayed a slower set and work times than EGDMA under the same conditions (FIGS. 13C-D). At 0.5 wt. %, BDMA's work time and set time was 5 and 7.5 minutes, respectively, with a further decrease to 1 and 2 minutes at 1.0 wt. %. PFDMA had similar work and set times to BDMA with (0.1 wt. %: 6 and 7 minutes, 1 wt. %: 1 and 1.3 minutes) (FIGS. 13E-F). G′ is a storage modulus (representative of elastic or solid behavior of a fluid) and G″ is a loss modulus (representative of inelastic or liquid behavior of a fluid). All of the 5 wt. % compositions cured before measurements could be taken with the rheometer (<30 seconds). The findings demonstrate the utility of the redox system to increase the cure rate of the polyHIPEs to ranges comparable to PMMA bone cements (e.g., which transitions from a low viscosity liquid to a rigid solid within 15 minutes). The findings show that a practitioner can work with either a liquid or putty to best suit their needs. Furthermore, the cure rate could be modulated. In these examples, modulation was from about 30 seconds to 10 minutes. In these examples, the modulation occurred by changing the redox initiator concentration. This is contrasted with PMMA, which is non-porous, non-biodegradable, and highly exothermic (peak temperatures reaching 110° C. or more). The described polyHIPEs cured to form porous and degradable materials with a maximum exotherm of only 42° C. The low exotherm is critical when these described materials are provided as scaffolds and used synergistically with living cells, small organisms, and/or bioactive components (e.g., growth factors), some examples of which will be discussed further below.

Additional rheological and gel fraction data were analyzed to investigate the impact of redox initiation concentration on network formation in candidate polyHIPEs. In each material, an induction period was evident prior to an increase in modulus that was dependent on macromer chemistry (EGDMA<BDMA<PFDMA). The induction period appeared to be primarily responsible for the difference in cure times, as depicted in FIGS. 13A-F. Without being bound by theory it is hypothesized that reduced radical diffusion and steric hindrances associated with increased macromer molecular weight resulted in longer induction periods, especially in the low initiator concentration compositions. PFDMA, BDMA, and EGDMA have molecular masses of approximately 362, approximately 226, and approximately 198 g/mol, respectively. HIPE viscosity trended with macromer molecular weight with PFDMA almost 30 times more than EGDMA. The increased viscosity likely inhibited initiator diffusion and reaction in the PFDMA HIPEs as compared with EGDMA HIPEs. This induction period decreased and the moduli slopes increased as initiator concentration was raised from 0.5 to 1.0 wt. %, suggesting increased polymerization rate. This was attributed to increased initiation sites leading to more chains growing simultaneously and causing chain molecular weight to increase more rapidly.

Gel fraction was used to compare an extent of network formation in polyHIPEs after 24 hours of curing. Network formation ranged from 78% to 92% for all compositions, depicted in TABLES 3 and 4. As expected, increasing initiator concentration correlated with increased gel fraction. PFDMA gel fraction increased the most, from 78% to 86%. BDMA gel fraction also increased significantly with higher initiator concentrations (86% to 92%). EGDMA gel fractions increased from 86% to 89%. Both EGDMA and BDMA had significantly higher gel fractions than the corresponding PFDMA polyHIPEs, likely due to steric hindrance and reduced radical diffusion associated with its higher molecular weight. Additionally, it is possible that highly crosslinked microgels, when formed, begin to sterically hinder further crosslinking, increasing network defects and free-ends. It should be noted that PGPR was not removed from the specimens prior to DCM incubation and this should account for approximately 9% of specimen mass. FTIR spectroscopy of the extract solutions showed the presence of PGPR, but the concentration was not quantified (data not shown). Assuming all of the PGPR was removed with the DCM, gel fractions were actually between 94% and 100%. Overall, these polyHIPEs showed excellent network formation that was further enhanced at higher initiator concentration. In TABLES 3 and 4: *=P<0.001 compared to EGDMA 1.0 and 5.0 wt. % pore sizes; †=P<0.05 compared to BDMA 0.5 and 1.0 wt. % pore sizes; ‡=P<0.01 compared to PFDMA 1.0 and 5.0 wt. % pore sizes; and =P<0.01 compared to 0.5:1.0 and 5.0:1.0 TMA:BPO pore sizes.

TABLE 3

| | | | | Interconnect |
|---|---|---|---|---|
| | Redox Initiator | Gel Fraction | Pore Diameter | Diameter |
| Material | (wt. %) | (%) | (μm) | (μm) |
| EGDMA | 0.5 | 86.1 ± 2.8 | 27 ± 12 * | 3 ± 2 |
| | 1.0 | 89.1 ± 0.5 | 20 ± 10 | 3 ± 1 |
| | 5.0 | 89.0 ± 1.0 | 19 ± 12 | 3 ± 1 |
| BDMA | 0.5 | 85.8 ± 0.4 | 14 ± 6 | 3 ± 1 |
| | 1.0 | 89.3 ± 0.4 | 14 ± 6 | 3 ± 1 |
| | 5.0 | 92.1 ± 0.3 | 13 ± 7 † | 2 ± 1 |
| PFDMA | 0.5 | 77.9 ± 1.7 | 5 ± 3 ‡ | 1 ± 1 |
| | 1.0 | 81.0 ± 0.8 | 6 ± 3 | 1 ± 1 |
| | 5.0 | 85.6 ± 0.8 | 6 ± 3 | 1 ± 1 |

TABLE 4

| | | | | Interconnect |
|---|---|---|---|---|
| | TMA:BPO | Gel Fraction | Pore Diameter | Diameter |
| Material | (wt. %:wt. %) | (%) | (μm) | (μm) |
| EGDMA | 0.5:1.0 | 87.8 ± 0.7 | 19 ± 8 | 3 ± 1 |
| | 1.0:1.0 | 89.1 ± 0.5 | 20 ± 10 • | 3 ± 1 |
| | 5.0:1.0 | 86.5 ± 1.9 | 17 ± 11 | 2 ± 1 |

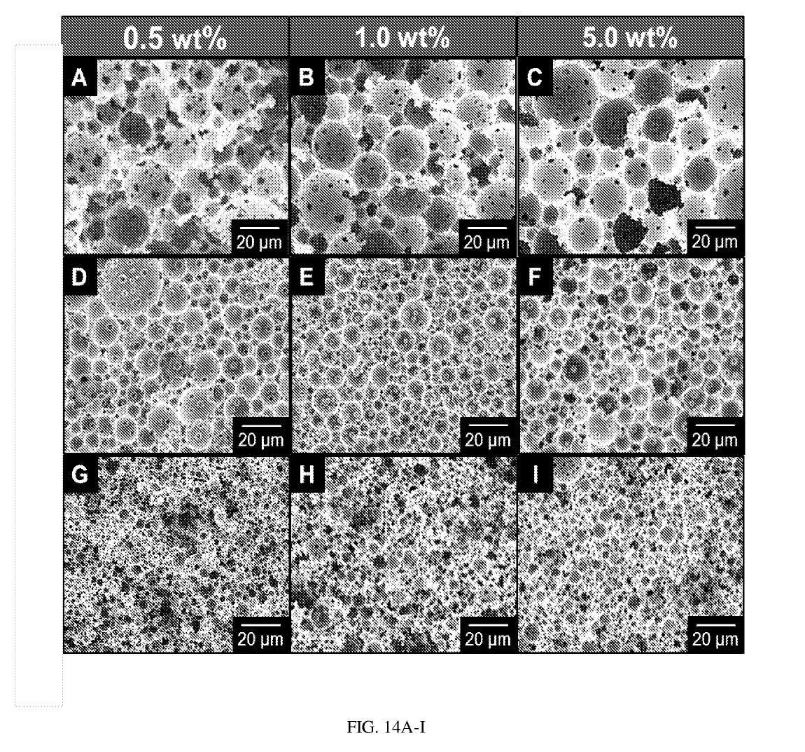

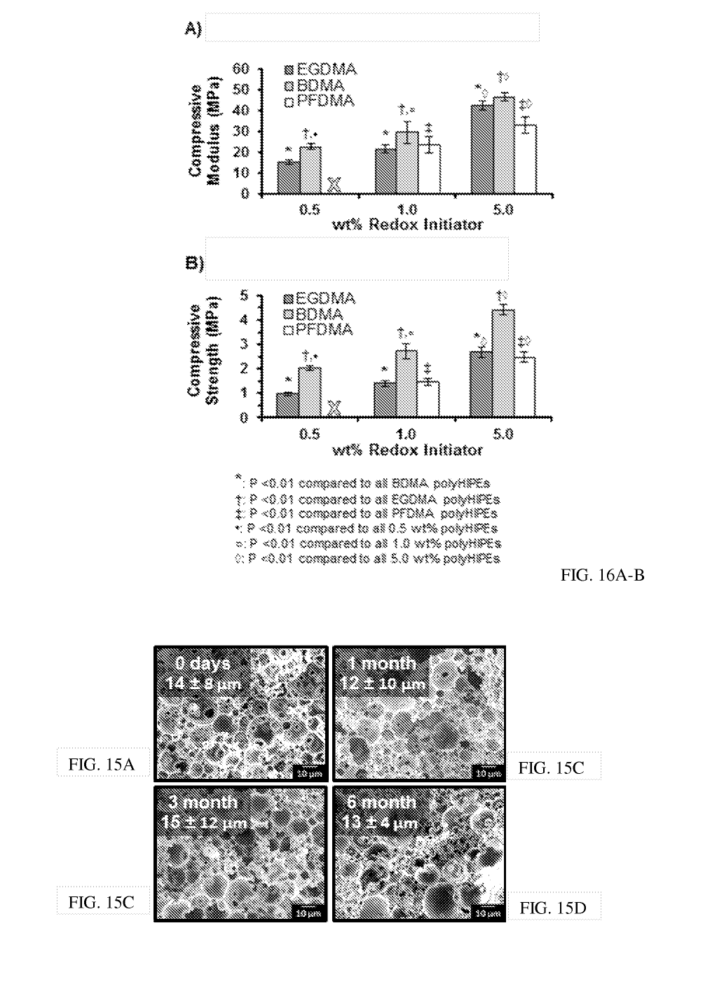

The relationship between the rapid, redox-initiated cure and polyHIPE micro-architecture was examined. First, it was found that desirable pore size and interconnection was retained. In the representative examples, EGDMA polyHIPEs possessed the largest pore diameters, almost double the size of BDMA and quadruple the size of PFDMA pores at each initiator concentration. Traditionally, pore size has been used as a marker of emulsion stability with smaller pore size indicating enhanced stability and reduced droplet coalescence prior to the gel point. Here, increased pore size was found to correlate with decreasing HIPE viscosity: PFDMA (11.0 Pa*s), BDMA (0.464 Pa*s) and EGDMA (0.343 Pa*s). Pa*s is the viscosity measure in Pascal seconds. Despite differences between materials, scanning electron micrographs revealed that average pore and interconnect diameter were generally not affected by redox initiator concentration for most tested materials (FIGS. 14A-I; see also TABLES 3 and 4). The 0.5% EGDMA was the exception with an average pore size of 26 μm, significantly larger than the 1.0 and 5.0% polyHIPEs (which was 20 μm), suggesting that some coalescence may have occurred prior to the gel point. The rapid cure of both the 1.0 and 5.0 wt. % EGDMA polyHIPEs reduced droplet coalescence and thus no change in pore size was observed. Thus, emulsion composition and its processing can be modified to increase or decrease pore diameter. And, recent studies in cardiac and skeletal tissue remodeling or regeneration indicate that pores that are <40 μm pores improve tissue regeneration. BDMA and EGDMA polyHIPEs are generally capable of being designed to form pore diameters up to about 60 μm when cured via thermal initiation (data not shown). The use of the static mixing head (and/or its diameter) may be used to decrease pore size. Without being bound by theory, the mixing head may be used to impart extra shear forces on the emulsion and further break droplets down to smaller diameters. Therefore, use of a larger or smaller diameter mixing head may be selected, as desired, when designing the final architecture, in order to help adjust pore diameter, pore shape and overall morphology.

With use of a double-delivery system, such as a double-barrel device, or a similar type mechanism or device, two HIPEs may be stored separately until needed. This extends shelf-life of the described HIPEs, especially since said HIPEs may be stored at reduced temperatures. HIPEs can be stored at least at 4° C., or at lower temperatures. In an example, various PFDMA HIPE samples were stored at about 4° C. and removed and cured (polymerized to form polyHIPEs) at various time points, including 1 month (FIG. 15B), 3 months (FIG. 15C) and 6 months (FIG. 15D). No observed effect on pore size (e.g., droplet coalescence) was observed, nor was there any phase separation over time as compared with polyHIPEs prepared at time zero (FIG. 15A). Overall, there was no observable or significant change in pore architecture after 6 months of storage. Thus, the described solvent free emulsions may be stored for at least six months without concern. The polyHIPEs may also be formed and cryogenically preserved. This is especially beneficial when polyHIPEs are formed and encapsulate live cells or other biologic components or therapeutics, suggesting there will be no change in efficacy over time or with storage. Various production levels are therefore acceptable for the claimed methods and systems. Emulsion-filled systems should be well suited for large scale production, transport and long-term storage.

The polyHIPEs described herein set within several minutes, and were further characterized on sections taken, generally after a 24 hour cure time. Compressive modulus and strength were found to increase as redox initiator concentration increased for all representative materials tested, as depicted in FIG. 16A-B. BDMA was significantly stiffer and stronger than both EGDMA and PFDMA for each concentration tested. Differences in strength were also found in the modulus. For example, the weakest BDMA (0.5 wt. % initiator) had a higher yield strength than all but the strongest EGDMA and PFDMA polyHIPEs (5.0 wt. % initiator). Some 0.5 wt. % PFDMA samples had a lower gel fraction and were not tested. Without being bound by theory, a high viscosity of PFDMA is thought to decrease mixing efficiency of the two HIPEs and limit radical diffusion resulting in regions of uncured HIPEs. This was not observed in the 1.0 or 5.0% PFDMA HIPEs, likely due in part to the higher concentration of initiator which limited the role of radical diffusion throughout the material. It is proposed that a longer static mixing head may eliminate any uncured regions, such as in a 0.5% redox PFDMA composition.

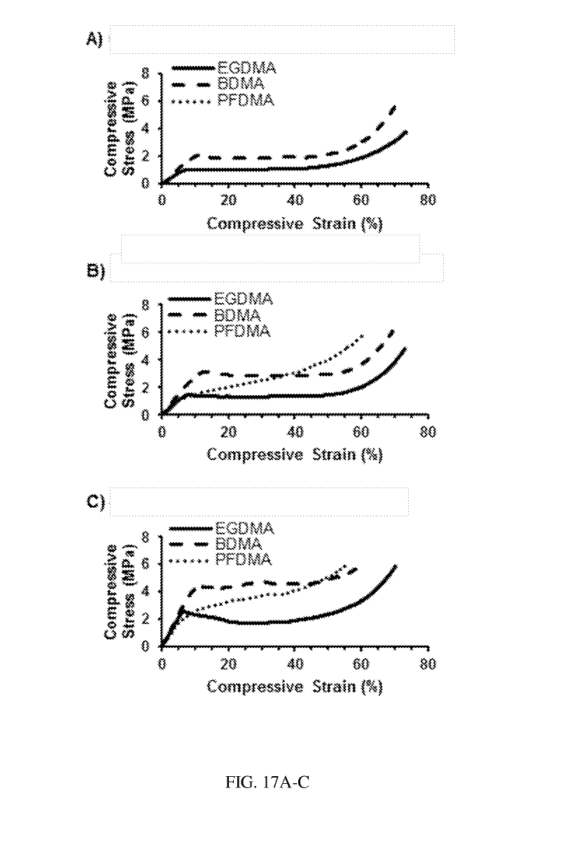

Representative compressive loading curves are presented for various representative materials at differing redox initiator concentration (see FIGS. 17A-C). Some EGDMA polyHIPEs were brittle and reduced to a compacted powder after compressive testing. BDMA and PFDMA polyHIPEs retained their dimensions and could be suitably loaded. Toughness for all the representative materials appeared to increase at higher initiator concentrations. BDMA and PFDMA specimens with 5 wt. % redox initiators showed no signs of brittle fracture. Compressive modulus and strength are clinically important, especially for tissue grafts, such as bone grafts when stabilizing a defect. Increased defect stability in bone reduces the necessity for immobilization and allows for earlier loading which has established benefits in stimulating bone regeneration.

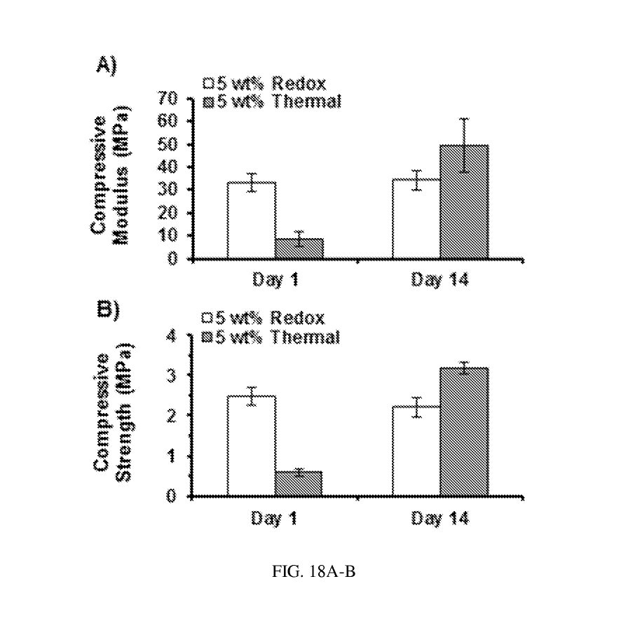

Overall, it was found that these porous materials, when formed as a monolith, had compressive properties that approached that of cancellous bone when matched by density. Thus, said materials may be used to mechanically stabilize a defect and elicit the appropriate mechanical cues to regenerate a tissue such as bone. Since some studies have shown that the mechanical properties required to trigger bone formation may be much lower than those of fully matured bone tissue, these materials may be suitably manipulated as desired. It was also found that with the described redox initiator system used herein, there was a rapid maturation of mechanical properties as compared to thermal initiation alone (FIGS. 18A-B). The compressive modulus and strength of PFDMA polyHIPEs that were thermally cured (e.g., with 5 wt. % BPO) required two weeks to reach maturation (see increase over a 2 week incubation at 37° C., dark bars). In that time, with thermal curing, modulus changed from 8.5 MPa to 43 MPa and strength changed from 0.4 MPa to 3 MPa. In contrast, the compressive modulus and strength of PFDMA polyHIPEs that were cured as described herein (e.g., with the described 5 wt. % redox initiator, was near maximal after 24 hours of incubation, and remained constant after 2 weeks. Accordingly, the redox system described herein is immediately available for loading. Said polyHIPEs when formed may serve as alternative to known fixation device, since they allow for nearly immediate loading after forming, or at least within 24 hours after introduction in vivo.

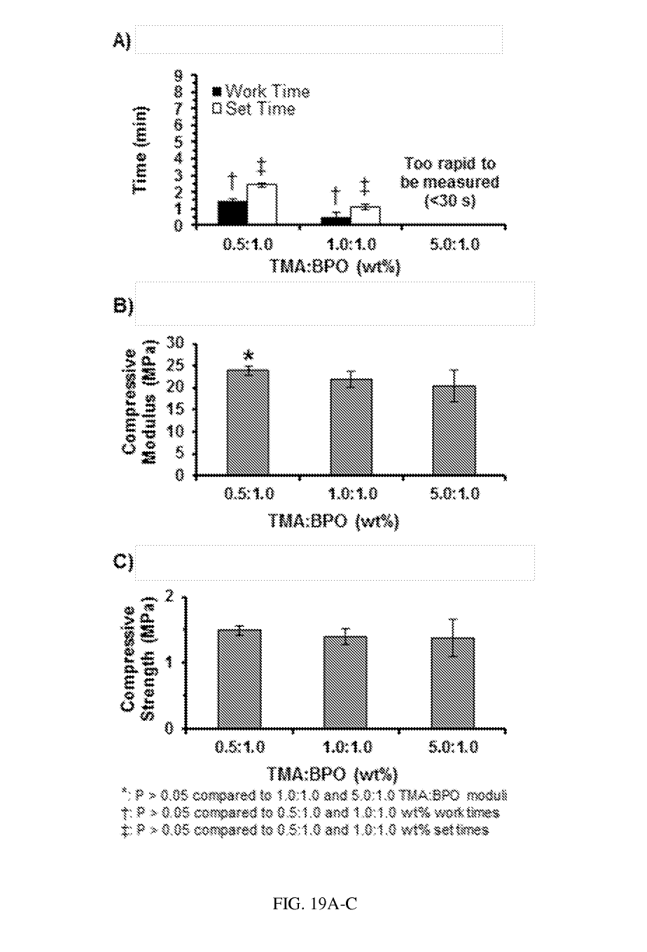

The ratio of reductant component to oxidant component was further investigated. In short, as expected, increasing the relative amount of TMA to BPO resulted in decreased work and set times, e.g., from 90 to 30 seconds (work time) and 2.5 to 1 minute (set time) (FIG. 19A, using EGDMA as an example). The 5.0 wt. % TMA:1.0 wt. % BPO HIPE actually set before the measurement could begin (<20 seconds). Without being bound by theory, it is found that increasing the relative amount of TMA increases its availability to react with BPO, resulting in faster radical production and initiation. The formation of the BPO-TMA complex may be used as a rate limiting step in radical production. Thus, rate if formation may be tailored as appropriate. A faster initiation allows the described HIPEs to form a network more quickly and to increase the cure rate. Compressive data collected after 24 hours indicated that redox initiator ratio had little to no effect on actual compressive modulus and strength (FIGS. 19B and 19C, respectively). There was also minimal observed effect on pore architecture. While, in some instances, an average pore diameter varied slightly as the relative amount of TMA was increased, there was no observable trend. The 1.0:1.0 ratio had the largest average pore diameter (20 μm) and the 0.5:1.0 and 5.0:1.0 were found to exhibit slightly smaller pore diameters at 19 μm and 17 μm, respectively. Overall pore architecture appeared similar between the compositions.

The above findings demonstrate that polyHIPE work and set time may be tuned independently from other polyHIPE properties (compressive modulus/strength, pore or interconnect diameter, architecture) with small variations in the reductant:oxidant ratio. Thus, when the described polyHIPE compositions are provided as a device, or as a tissue engineered graft, there is the opportunity to tune physical properties, to adjust for cytocompatibility and to optimize work and set time to meet clinical demands and/or preferences.

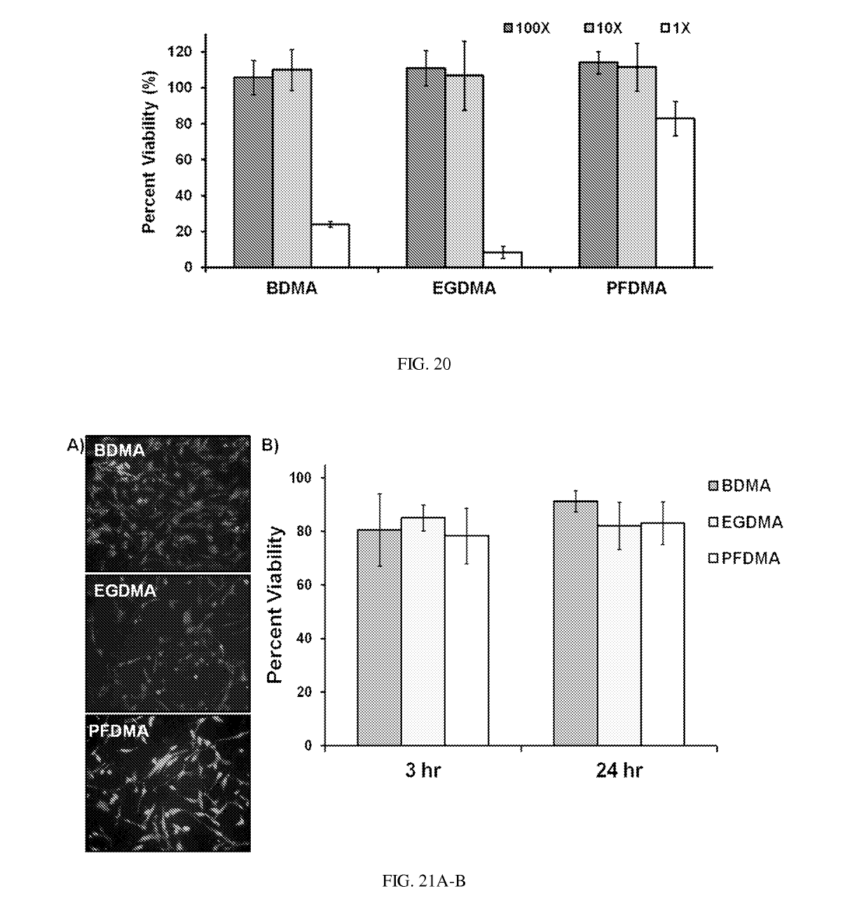

The described compositions did not reveal cytotoxic effects. For example, in studies analyzing the surfactant PGPR, there was no apparent direct cellular exposure of the surfactant at any of the concentrations described (>95% viability). Assessment of hMSC viability was also performed after exposure to extract media from unreacted macromers and direct seeding onto cured polyHIPE grafts. This provides an assessment as to whether there is an effect from exposure of the described macromer to cells prior to crosslinking in situ. Cell viability of hMSCs after exposure to PFDMA extract media was high (>80%)(FIG. 20). Cell viability was less after exposure to EGDMA or BDMA extract media. With dilution of the same extract media by 10 fold and by 100 fold, the viability of all cells remained high, regardless of which macromer the cells were exposed to (FIG. 20). Furthermore, direct seeding of cells on crosslinked polyHIPE structures was also evaluated. This provided evidence that the crosslinked polyHIPEs, when formed were capable of forming support structures, by promoting both hMSC adhesion and viability. Cell viability results for all three macromers were greater than 80% at 24 hours, as depicted in FIGS. 21A-B. These results provide sufficient evidence that all the macromers can be considered cytocompatible. And, since degradation products of these polyHIPE compositions are considered to be similar to the initial composition, the biodegradable by-products of the described polyHIPE structures are also not anticipated to be detrimental.

Cells delivery via the described polyHIPE composition delivery system is also disclosed. This allows viable cells (e.g., autologous hMSCs) to be delivered to a desired physiologic site (e.g., defect or damage site), promoting healing and reducing cell or immune rejection, often found when allogeneic tissues are delivered.

Cells are encapsulated in the polyHIPE upon injection, meaning that upon curing, the cells remain encapsulated in the rigid, highly porous foam when said polyHIPE is fully cured. This overcomes problems associated with mechanical weakness in typical hydrogel systems. The injectable polyHIPEs when formed create foam bodies that serve as a protective substrate for delivery of cells or other bioactive or biologic materials. Furthermore, the rigid foam bodies, as shown above, can withstand physiological loading conditions.

To ensure encapsulation and maintenance of cell viability it has been found that the described injectable polyHIPEs offer both adequate viscosity for proper mixing with a cell suspension and a quick cure time, which reduced time for diffusion of unreacted macromer and radicals into the cell-laden aqueous phase or the diffusion of cells away from the emulsion. As depicted in FIGS. 22A and 22B, double delivery systems suitable for encapsulating cells are disclosed. In this system a first delivery unit contains the HIPE emulsion (comprising all the required components, including the redox initiators) and a second delivery unit contains the cells. In some embodiments, the first delivery unit has at least twice the volume of the second delivery system. In some embodiments, the first delivery unit has more than twice the volume and up to ten times or fifteen times or twenty times the volume of the second delivery unit.

In one example, a BDMA HIPE with 0.5 wt. % BPO and 2.5 wt. % ferrocene was fabricated with basal media as the aqueous phase and loaded in the larger side of a 10:1 double barrel syringe. Cells (hMSCs) at a fifth passage were trypsinized and loaded into the smaller unit in a suspension to ensure a 10:1 ratio. This head was of a larger diameter and length to ensure proper mixing of the HIPE components with the cell suspension. It is possible that a further decrease in the viscosity of the organic phase may allow the use of a 1:1 double barrel while maintaining successful mixing of the two phases.

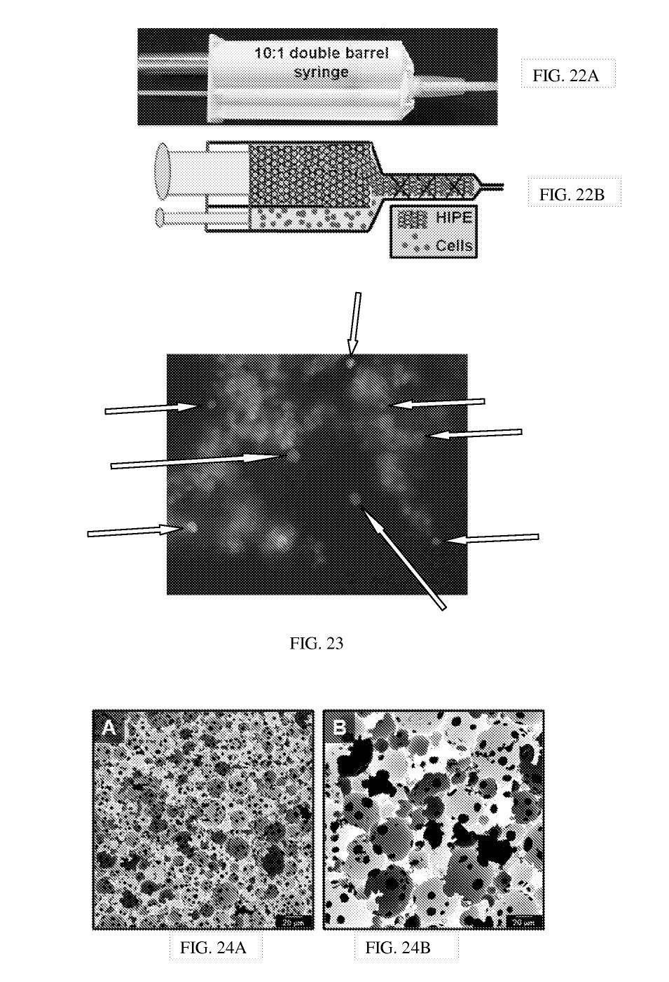

A representative micrograph of hMSC viability in sections of the BDMA polyHIPE structure when formed is shown in FIG. 23. On observation it was found that a majority of the cells remained viable post encapsulation. Many hMSCs were visible throughout all sections illustrating the ability to adequately mix the cell suspension with the HIPE and to provide an increased distribution within the monolith.

The pore architecture after cell encapsulation is illustrated in FIGS. 24A and 24B. Some alterations in overall morphology and porosity were observed. In the example, there were, on average, larger, homogenous pore sizes (20-100 μm) and larger interconnect sizes (5-10 μm). These values should be able to be readily modulated by choice of the HIPE macromer, the emulsion composition (e.g., redox initiators and amounts), as well as the number of cells and/or components in the media. Where increased nutrient and waste transport is desired, increased interconnected polyHIPE pore size can be prepared throughout the monolith.

PolyHIPE compositions that are cytocompatible and can be manipulated to polymerize rapidly, as desired, and methods for making said polyHIPE compositions are disclosed. These polyHIPE compositions polymerize via a redox reaction, are fast curing, and can be suitably controlled to optimize cure time, porosity, pore shape, and/or hardness. Said polyHIPE compositions are capable of curing ex vivo or in vivo, are deliverable to a specific site, and are biodegradable. The polyHIPE compositions may be used in vivo and are also amenable for use in other applications requiring a porous polymeric material.

A low temperature polymerization as described herein allows the compositions to form in vivo or ex vivo, and when formed to be applicable for use in vivo or ex vivo. Moreover, the compositions described herein may incorporate one or more additional components prior to polymerization, including highly sensitive components (biologic or non-biologic materials), including but not limited to cells, growth factors, enzymes, and proteins. It is also understood that while the preparations and polymerizations described herein may occur at physiologic temperatures, they may also occur at other temperatures, even at a temperature that is up to or higher than 100° C.

As described, the combined initiators described herein (redox-initiator) when combined with the biodegradable HIPEs described herein and formed via a double delivery system provide suitable polyHIPE compositions for use as a tissue scaffold, replacement, and/or graft. The combined initiators described herein reduced work and set times for the described polyHIPEs from hours to minutes, matching current cement products, such as PMMA. These reductions in cure times were achieved with lower total concentration of the initiator, which may be, in part, for the enhanced cytocompatibility of the described polyHIPEs.

Compressive modulus and strength may be manipulated with adjusting redox initiator concentration with minimal impact on pore architecture. Further modulation of the reductant:oxidant ratio decoupled set time from compressive modulus and strength allowing for increased tunability when forming an ex vivo, in vivo or in situ scaffold. The compressive modulus and strength values for the described formed rigid foam structures are higher than was is found in typical hydrogel matrices. Said values are comparable with values shown to be suitable for promoting healing.

Use of a double delivery system for introducing polyHIPEs ex vivo, in vivo or in situ, as described herein, also permits emulsions to be prepared and stored well in advance of use. Preparation and storage may be many months, and likely even a year prior to use. Storage may be at an ambient temperature. In some embodiments, storage is preferably at sub ambient temperatures, and reduced temperatures (e.g., at refrigerated temperatures, such as at or below 4° C.) to prevent possible slow curing. The double delivery system described herein also allows for curing at a rate that is less than two hours, such as in about 100 minutes, or less than about 100 minutes. Rapid curing may also be less than about 90 minutes, or less than about 60 minutes, or may be less than about 50 minutes, or may be less than about 45 minutes, or may be less than about 40 minutes, or may be less than about 30 minutes, or at or less than about 25 minutes, or at or less than about 20 minutes, at or less than about 15 minutes, or at or less than about 10 minutes, or at or less than about 5 minutes, or at or less than about 4 minutes, or at or less than about 3 minutes, or at or less than about 2 minutes, or at or less than about 1 minutes, or at or less than about 30 seconds, or any range there between. The in situ delivery of the described polyHIPEs provides an on-demand structure without sacrificing porosity or mechanical properties.

Morphology of the described polyHIPEs are well suited for cell adhesion and cell growth. Said morphology and architecture also translate into a support scaffold that permits physiologic loading.

In one or more embodiments, polymerizable HIPEs are injectable in situ for use in filling irregular bone defects. Said polyHIPEs when fully polyermized serve as rigid support structure having various degrees of porosity. Said structure as porous scaffolds may potentially improve tissue healing.

As described are HIPE compositions that remain stable without initiating polymerization or are capable of being prepared so that polymerization is significantly delayed (as compared with HIPE compositions comprised of similar macromers for polymerization). The HIPEs are prepared from starting biodegradable material (e.g., macromer) that forms a resulting biodegradable polymeric material with a specific and appropriate viscosity and hydrophobicity for emulsification when stabilized with an emulsifier. The macromer will also include certain structural features (e.g., ester or anhydride linkages; hydrogen bond acceptor sites; unsaturated end groups).

In the claims, the word “comprising” does not exclude other elements or steps, and the indefinite article “a” or “an” does not exclude a plurality. The mere fact that certain measures are recited in mutually different dependent claims does not indicate that a combination of these measured cannot be used or used to an advantage.

While the invention has been illustrated and described in detail in the drawings and foregoing description, such illustration and description are considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

1. A high internal phase emulsion comprising:
biodegradable polymeric material, the biodegradable polymeric material comprising:
at least one end group selected from one or more of an acrylate and a methacrylate; and
one or more linkages selected from one or more of an anhydride and an ester,
the biodegradable polymeric material having an octanol-water partition coefficient in a range from about 2 and about 8, and a viscosity in a range from about 0.08 cP and about 1000 cP,
the biodegradable polymeric material being stabilized with a quantity of an emulsifier lacking hydrogen bond donors in a hydrophilic head region while having a hydrophilic-lipophilic balance in a range from about 2 and about 9,
a chemical to prevent Ostwald ripening, wherein the chemical to prevent Ostwald ripening is a salt;
an oxidizing agent in a quantity that maintains the high internal phase emulsion as an emulsion and is insufficient to initiate extensive free radical cross-linking of the biodegradable polymeric material when stored at sub-ambient temperatures; and
water, wherein the water is in a volume of at least about 75% of the high internal phase emulsion by volume,
wherein the high internal phase emulsion is substantially stable as an emulsion without undergoing significant polymerization when stored at a sub-ambient temperature.

2. The high internal phase emulsion of claim 1, wherein the octanol-water partition coefficient of the biodegradable polymeric material is in a range from about 2 and about 4.

3. The high internal phase emulsion of claim 1, wherein the viscosity of the biodegradable polymeric material is near a viscosity of water.

4. The high internal phase emulsion of claim 1, wherein the oxidizing agent is a free radical oxidizing initiator.

5. The high internal phase emulsion of claim 1, wherein the hydrophilic-lipophilic balance of the emulsifier is in a range from about 3 and about 5.

6. The high internal phase emulsion of claim 1, further comprising:
a reducing agent.

7. The high internal phase emulsion of claim 6, wherein the octanol-water partition coefficient of the biodegradable polymeric material is in a range from about 2 and about 4.

8. The high internal phase emulsion of claim 6, wherein the viscosity of the biodegradable polymeric material is near a viscosity of water.

9. The high internal phase emulsion of claim 6, wherein the reducing agent is selected from one or more of a hydrocarbon, metal ion, vitamin, and bioactive agent.

10. The high internal phase emulsion of claim 6, wherein the hydrophilic-lipophilic balance of the emulsifier is in a range of from about 3 and about 5.

11. A method of making a high internal phase emulsion comprising:
combining a first high internal phase emulsion with a second high internal phase emulsion by dispersion;
wherein the first high internal phase emulsion comprises an oxidizing agent in a quantity insufficient to initiate extensive cross-linking in the first high internal phase emulsion when stored at sub-ambient temperatures, and
wherein the second high internal phase comprises a reducing agent.

12. The method of claim 11 further comprising:
combining the first high internal phase emulsion containing an oxidizing initiator with the second high internal phase emulsion containing a reducing agent by dispersion; and
allowing the first high internal phase emulsion and the second high internal phase emulsion to undergo a redox reaction;
wherein upon combining a foam body is formed, the foam body having a porosity at or greater than 75%.

13. The method of claim 12, wherein a rate of combining has an effect on an average pore diameter in the foam body.

14. The method of claim 12, wherein the foam body is injectable into any one or more of a form, a mold, and in situ, such that the foam body takes a shape of said form, said mold, or may be formed in situ.

15. The method of claim 12, wherein the foam body has a pore size in any size or range of sizes from about 1 micron and 300 microns.

16. (canceled)

17. A high internal phase emulsion composition comprising:
a first high internal phase emulsion comprising at least a first biodegradable polymeric material, an oxidizing agent, and water, wherein the oxidizing agent is in a quantity that maintains at least a portion of the first high internal phase emulsion as an emulsion and delays cross-linking of the first high internal phase emulsion when stored at sub-ambient temperatures, wherein the water is in a volume that is at least about 75% of the first high internal phase emulsion by volume; and
a second high internal phase emulsion comprising at least a second biodegradable polymeric material, a reducing agent, and water, wherein the water is in a volume that is at least about 75% of the second high internal phase emulsion by volume.

18. The high internal phase emulsion composition of claim 17 further comprising one or more of a bioactive component and a cell in an aqueous phase of any or both the first high internal phase emulsion and the second high internal phase emulsion.

19. The high internal phase emulsion composition of claim 17 further comprising a bioactive component in an organic phase of any or both the first high internal phase emulsion and the second high internal phase emulsion.

20. The high internal phase emulsion composition of claim 17, wherein the first high internal phase emulsion and the second high internal phase emulsion may be stored separately at sub ambient temperatures.

21. The high internal phase emulsion composition of claim 17, wherein the first biodegradable polymeric material and the second biodegradable polymeric material contain at least one end group selected from one or more of an acrylate and a methacrylate, and one or more linkages selected from one or more of an anhydride and an ester.
